(19) United States
(12) Patent Application Publication
(10) Pub. No.: US 2010/0280494 A1
(43) Pub. Date: Nov. 4, 2010

(54) CATHETER AND ARRAY FOR ANTICANCER THERAPY

(76) Inventors: James E. Matsuura, Fort Collins, CO (US); Stephen L. Warren, Fort Col, CO (US); Kevin Lillehei, Englewood, CO (US)

Correspondence Address: SCHWEGMAN, LUNDBERG & WOESSNER, P.A., P.O. BOX 2938, MINNEAPOLIS, MN, 55402, US

(21) Appl. No.: 12/375,583

(22) PCT Filed: Jul. 25, 2007
(86) PCT No.: PCT/US07/16701

§ 371 Date: Aug. 14, 2009

Related U.S. Application Data
(60) Provisional application No. 60/895,916, filed on Mar. 20, 2007.
(60) Provisional application No. 60/821,775, filed on Aug. 8, 2006.

(51) Int. Cl. A61M 25/00; A61M 36/06

(52) U.S. Cl. 604/523; 600/4

(57) ABSTRACT

A catheter array system adapted for implanting a plurality of catheters within the tissue of a patient in a spatially defined array, comprising a plurality of catheters, a catheter guide template adapted to guide the implantation of catheters, and a liquid supply system including a pressurizer and a manifold, is provided. A method of treatment of a malcondition in a patient comprises implantation of a spatially definted array of catheters using the system is also provided. The bioactive agent can be a radiotherapeutic agent, a chemotherapeutic agent, a protein, an antibody, an oligonucleotide-based therapeutic agent such as siRNA, or a combination of agents. A preferred radiotherapeutic agent is ¹²³I- or ¹²⁵I-IUDR, for example in the treatment of locally advanced tumors, such as glioblastoma multiforme.

### CLAIM OF PRIORITY TO RELATED APPLICATIONS

This application claims the priority of U.S. Patent Ser. No. 60/821,775, filed Aug. 8, 2006, and to U.S. Patent Ser. No. 60/895,916, filed Mar. 20, 2007, which are incorporated herein by reference in their entireties.

### BACKGROUND OF INVENTION

In the treatment of neoplasia, such as solid tumors in the early stages, surgical excision or ablation with radiation often provides a successful form of therapy. However, this is not the case for many solid tumors that have advanced to later stages. Locally advanced or locally invasive solid tumors are primary cancers that have extensively invaded or infiltrated into the otherwise healthy tissues surrounding the site where the tumor originated. Locally advanced tumors may arise in tissues throughout the body, but unlike early stage tumors may not be amenable to complete surgical excision or complete ablation using radiation treatments. Due to the invasion of the surrounding tissues by tumor processes, any surgical procedure that would serve to remove all the cancerous cells would also be likely to maim or destroy the organ in which the cancer originated. Similarly, radiation treatments intended to eradicate the cancerous cells left behind following surgery frequently lead to severe and irreparable damage to the tissues in and around the intended treatment field. Often, surgery is combined with radiotherapy, chemotherapy or a combination of adjuvant therapies designed to eliminate the malignant cells that could not be removed by the surgery. However, when a tumor has infiltrated into otherwise healthy tissues surrounding the site where the tumor originated, even combination treatments including surgery plus radiation therapy, or surgery plus radiation therapy plus chemotherapy may not be capable of eradicating the tumor cells without causing severe damage to the tissues in the treatment field.

In cases involving locally advanced tumors, surgery may be used for gross excision, a procedure referred to as “debulking,” but the surgeon at present does not have the tools to eliminate individual tumor cells, microscopic tumor processes, or tumor-associated vasculature from the normal tissue surrounding the tumor excision site. It is often critical to minimize the volume of surrounding tissue that is excised in such operations. For example, in the case of tumors of the central nervous system, normal brain functions may be severely compromised as a result of tissue loss. Thus, in such cases surgery is often accompanied by radiation therapy and/or chemotherapy in an attempt to kill cancerous cells remaining in the surrounding tissue. The chemotherapy may be delivered to the residual tumor cells by a localized or systemic route of administration. By limiting the extent of surgical excision, and relying upon the adjunctive treatments to eliminate the residual cancer cells, the function of an organ may be preserved.

Conventional radiation therapy, using ionizing radiation beams (X-ray, gamma ray, or high energy beta particles), while well-established as an anti-cancer treatment modality, is not curative in the majority of patients whose cancer is locally advanced. Another form of radiation treatment is brachytherapy, the implantation of sealed radioactive sources emitting gamma rays or high energy beta particles within the tissue adjacent to the tumor site, for example in treatment of brain or prostate cancer. For example, see U.S. Pat. Nos. 6,248,057, 6,743,211, and 6,905,455.

Even with the addition of systemic agents, nearly one third of patients with locally advanced solid tumors relapse (Vijaykumar, S. and Hellman, S., “Advances in Radiation Oncology,” Lancet, 349[S11]: 1-3 (1997)). Ionizing radiation, whether from a beam or from an isotopic implant emitting high energy radiation, lacks the specificity needed to eliminate the tumor cells while sparing the normal cells within the treatment field. Thus, collateral damage to normal tissues cannot be avoided. Conventional radiation therapy has several additional limitations. X-rays are administered by an intermittent schedule, usually 5 days per week, thereby providing an opportunity for the cancer cells to repair their DNA and to repopulate the tumor between treatments. Ionizing radiation requires sufficient oxygen in the tissues to eliminate tumor cells, but most solid tumors are relatively hypoxic, and therefore inherently resistant to radiation. In addition, the total lifetime dose of radiation is limited by the risk of severe late toxicities. Therefore, with few exceptions only a single treatment course, usually lasting no more than 6 weeks, can be administered to a tumor. Finally, ionizing radiation is itself oncogenic, especially when used in combination with chemotherapy agents.

Most types of chemotherapy also suffer from a lack of tumor specificity and also cause collateral damage to normal tissues, since chemotherapeutic agents are distributed throughout the body and exert their effects on normal cells as well as malignant cells. Many systemic chemotherapy agents act on cells undergoing DNA synthesis and cell division, and thus may impact many cell populations throughout the body in addition to the target cancer cells.

The deficiencies of current treatment modalities are especially glaring with respect to specific types of cancer, for example glioblastoma multiforme (GBM), a highly aggressive type of cancer that constitutes the most common form of brain malignancy. Indeed, after nearly 35 years of investigations involving hundreds of experimental treatments and thousands of GBM patients participating in clinical trials, the prognosis of patients with newly diagnosed GBM is dismal. In a recent survey, the survival following the diagnosis of GBM is only 42% at 6 hmonths, 18% at one year, and 3% at 2 years (Ohgaki, et al., “Genetic pathways to glioblastoma: A population-based study,” Cancer Research, 64:6892-6899 (2004)).

The currently favored treatment for newly diagnosed GBM is surgical resection followed by a course of ionizing radiation plus oral temozolomide, a chemotherapy agent that is administered during and after the course of radiation. In patients receiving this treatment, the best currently available, the median prolongation of survival is only about 2-3 months beyond surgery and radiation alone.

Recently, techniques have been developed to increase the effective concentration of chemotherapeutic agents at a tumor site. In the treatment of GBM, interstitial or localized chemotherapy has been used with modest success. Wafers containing carmustine (a chemotherapy agent) are inserted into the cavity created by surgical removal of the tumor. The wafers release carmustine into the brain tissue in the immediate vicinity of the brain tumor. This treatment has been shown to increase the median survival from 11.6 months to 13.9 months in patients also treated with surgery and radiation beam therapy (Westphal, M., et al., “A phase III trial of local chemotherapy with biodegradable carmustine (BCNU) wafers in patients with primary malignant glioma,” Neuro-oncology, 5:79-88 (2003)). Interstitial chemotherapy may be particularly well suited for treatment of GBM, as greater than 90% of GBM tumors that recur following surgical resection are localized within 2 cm of the surgical margin (Hochberg, F. H., and Pruitt, A., Neurology, 30:907-911 (1980)).

Localizing the concentration of the chemotherapeutic agent by physical techniques (as distinct from biochemical targeting) thus seems to offer certain advantages compared to systemic chemotherapy, as shown by the encouraging results with carmustine wafers. However, the challenge is great, because the majority of chemical entities do not diffuse far into brain tissue or other types of solid tissues.

Another development in physically localized delivery of chemotherapeutic agents is convection enhanced delivery. In this technique as applied to brain tumors, a fluid is delivered directly to a site in the brain and not through the circulatory system. The fluid is applied under sustained pressure such that the liquid moves through the interstices of the tissue, carrying with it any dissolved materials. For example, see Bobo, R. H., et al., “Convection-enhanced delivery of macromolecules in the brain,” Proc. Nat. Acad. Sci. USA, 91: 2076-2080 (1994); Laske, D W. et al. “Convection-enhanced drug delivery,” U.S. Pat. No. 5,720,720 (Feb. 24, 1998); and Hall, W. A., et. al. “Convection-enhanced delivery in clinical trials,” Neurosurg. Focus, 14(2), 1-4, (2003). Convection-enhanced delivery thus serves to increase the effective distance over which a bioactive agent can be delivered in solid tissue.

Convection-enhanced delivery usually involves the use of 3-5 catheters that are individually implanted directly into the brain tissue surrounding a surgical cavity created at the time of tumor removal. The catheters are inserted from multiple points of origin on the outer surface of the brain and not from within the brain tumor cavity. A pump propels the treatment fluid into the catheters, and therefore, bulk flow originates from the tips of the catheters. One of the biggest challenges associated with this type of drug delivery is to determine the optimal position for the catheter tips. Optimal positioning of catheter tips is important not only to ensure that the infusate gains access to the entire intended treatment field, which may be extensive and irregularly shaped, but also to minimize exposure to uninvolved regions of the brain. Other challenges are to provide sufficient coverage of the treatment field using a small number of catheter tips; to avoid backflow of the infusate around the catheter and back onto the surface of the brain; and to prevent the leakage of infusate into the cerebral ventricles and other anatomical sites of the brain.

The effective treatment of locally advanced solid tumors, including GBM, requires not only improved methods of drug delivery, but also therapeutic agents capable of eliminating the cancer cells while at the same time sparing normal tissues that have been invaded by the cancer cells. In this regard, a major issue revealed by studies of gene expression profiling, is that tumors are genetically and metabolically much more heterogeneous than previously anticipated. Tumors may be genetically and metabolically heterogeneous despite a common organ or tissue of origin, and despite a very similar appearance under the microscope. This is especially true of GBM and other malignant gliomas that arise in the central nervous system. For example, see Mischel, P. S. Cloughesy, T. F. and Nelson, S. F., “DNA-Microarray Analysis of Brain Cancer: Molecular Classification for Therapy,” Nature Cancer Reviews, 5:782-792 (2004). In view of the tumor heterogeneity, biochemical targeting, i.e. the search for agents that specifically target each tumor type, is a daunting challenge.

New and effective treatments are needed to: (a) eliminate tumor cells, including the tumor stem cell subpopulation, within the treatment field; (b) eliminate tumor cells with a wide range of genetic and metabolic profiles; (c) eliminate tumor stem cells with inherent resistance to chemotherapy and ionizing radiation; (d) minimize or avoid toxicity to normal cells and tissues. One approach to this problem is physically localized delivery of an agent capable of killing many different types of cancer cells, while at the same time having minimal or no toxicity to normal cells within the treatment field. This approach is distinct from the concept of targeted therapy, in which a different drug mechanism may be needed to treat each tumor according to its distinct genetic and metabolic profile.

A unique cell killing mechanism that has garnered considerable interest is the release of Auger electrons. These electrons are emitted by radionuclides that decay by electron capture and internal conversion. Examples of Auger emitting radionuclides include ¹²³Iodine, ¹²⁵Iodine, ⁷⁷Bromine and ^{80m}Bromine. Auger electrons have energies even lower than the energy of the beta particle emitted by tritium. This effect is amplified, because some Auger emitters release multiple electrons with each nuclear transformation. The low energy of the Auger electrons results in extremely short particle path lengths within tissues, which is highly desirable, because it minimizes collateral damage.

One molecular entity incorporating ¹²⁵I is [¹²⁵I]-iodouridine-deoxyriboside (¹²⁵IUDR), a thymidine analog. ¹²⁵IUDR is recognized by DNA polymerases as thymidine, and thus is incorporated into the chromosomes at times of DNA synthesis. Once incorporated into the DNA, the Auger electrons, with their very short range, have access to the chemical backbone of the DNA double helix. When the ¹²⁵I atom disintegrates, Auger electrons cause irreparable destruction of the chromosomes within the target cell, but with minimal effect on cells in the immediate vicinity of the target cell. ¹²⁵IUDR and related compounds destroy cells that make DNA, but have little or not effect on other cells.

Despite the recognition that ¹²⁵IUDR has a unique cell killing capability, and despite many years of research aimed at exploiting this mechanism of action, including the concept of directly introducing ¹²⁵IUDR into tumors (for example, see Kassis et. al., “Treatment of tumors with 5-radioiodo-2′-deoxyuridine,” U.S. Pat. No. 5,077,034), these agents have not been successfully applied to the treatment of cancer. The delivery of ¹²⁵IUDR and related agents to solid tumors, using systemic or local administration, has proven to be extremely challenging.

The effectiveness of incorporation of an Auger electron emitting nucleotide analogue into DNA during DNA synthesis may be increased by increasing the proportion of target cells engaged in DNA synthesis. This general approach has been used successfully to enhance the effects of numerous anticancer agents, particularly cytotoxic drugs that act preferentially on cells during S-phase of the cell cycle (i.e. “S-phase active agents”). For example, see Chu E. and DeVita. “Principles of Medical Oncology”, pp 295-306 in Cancer Principles and Practice of Oncology 7^{th} edition. Lippincott Williams & Wilkins© 2005. Certain drugs can block the progression of tumor cells during S-phase, thus effectively increasing the fraction of susceptible cells within the target cell population. This approach has been used successfully using a cell cycle inhibitor, 5-fluorouridine 2′deoxyribonucleoside, to increase the uptake and incorporation of 5-[¹²⁵I]-iodouridine 2′deoxyribonucleoside into DNA. For example, see: Holmes, J. M. The toxicity of fluorodeoxyuridine when used to increase the uptake of 125I-iododeoxyuridine into tissue culture cells in vitro. J. Comp. Pathol. 93:531-539 (1983); F. Buchegger et. al. Highly efficient DNA incorporation of intratumourally injected [¹²⁵I]iododeoxyuridine under thymidine synthesis blocking in human glioblastoma xenografts. Int J Cancer 110:145-149 (2004); and Perillo-Adamer, F. Short fluorodeoxyuridine exposure of different human glioblastoma lines induces high-level accumulation of S-phase cells that avidly incorporate ¹²⁵I-iododeoxyuridine. Eur J Nucl Med Mol Imaging 33: 613-620 (2006).

Thus, there is a need for new drug delivery devices and methods of use aimed at exploiting the unique mechanism of action of ¹²⁵IUDR and related compounds. New approaches are needed to deliver ¹²⁵IUDR (and other compounds) to solid tumors with the intent to eliminate cycling tumor cells, including the tumor-maintaining stem cells and their progenitors, while at the same time sparing normal tissues that have been invaded by the cancer cells. This need includes methods for delivery of such agents directly into the tumors and into the normal tissues that have been invaded by tumor cells, particularly in a way that provides for substantially uniform treatment of an often-irregularly shaped volume of tissue.

### SUMMARY

The present invention is directed to an apparatus and a method for delivery of bioactive agents, such as anticancer agents, to a target tissue such as brain tissue of a patient in need thereof. An embodiment of the invention provides a catheter array system for delivery of a liquid solution of a bioactive agent into a target tissue of a patient; the system comprising: a plurality of biocompatible catheters, each catheter comprising a linear or curvilinear hollow tube and being adapted for insertion into the body tissue, for remaining within the tissue for a period of time, and for delivery of the solution of the bioactive agent through the tube into the tissue; a catheter guide template adapted for guiding emplacement of each of the plurality of catheters into a tissue adjacent to the guide template to form a spatially defined catheter array within the tissue; and a pressurized liquid supply system adapted for delivery of a liquid via a manifold to each of the catheters; wherein each catheter comprises a distal portion for insertion into the tissue, at least one port whereby the solution can pass from inside the hollow tube into the tissue, a median portion adapted to be directed for insertion into the tissue by the guide template, and a base portion adapted for connection to the manifold of the pressurized liquid supply system; the catheter guide template comprises a plurality of catheter guideway channels, each guideway channel being adapted to guide movement of one or more catheters through the channel for insertion into the tissue such that upon insertion of the plurality of catheters, the catheters can form the spatially defined catheter array within the tissue; and the liquid supply system comprises a pressurizer adapted to apply a pressure to the liquid solution and a manifold to deliver the liquid under pressure to the base portion of each of the plurality of catheters such that the liquid can pass through the hollow tube of each catheter into the tissue.

Embodiments of the present invention further provide a catheter, a catheter guide template, and a liquid supply system including a pressurizer and a manifold, each of which adapted to be used as a component of the inventive catheter array system.

An embodiment of the present invention is directed to an array of catheters, disposed within a tissue of a patient in need thereof. The catheter array is preferably regular, wherein the catheters are'disposed in a parallel or a radial three-dimensional arrangement. The catheters are preferably spaced closely enough together that the distance between them is no greater than about twice the distance over which the bioactive agent can therapeutically penetrate the tissue. The catheters making up the array may be emplaced individually, in subsets of the total number, or all at once. Subsets of the catheter array can be implanted at different depths, and in different spatial arrangements within the tissue. The catheter guide template directs the formation of the spatially defined catheter array during the process of insertion of the plurality of catheters, which can take place sequentially, simultaneously, in subsets of the plurality of catheters.

The bioactive agent, a solution of which is introduced into the target tissue by the inventive catheter array system, may be a radiochemical, chemotherapeutic agent or other small molecule, antibody, protein, peptide, oligonucleotide aptamer, antisense oligonucleotide or a small interfering RNA (siRNA). One such radiochemical comprises an Auger electron emitter, such as ¹²³I- or ¹²⁵I-iodouridinedeoxyriboside (¹²³IUDR or ¹²⁵IUDR), wherein the radionuclide is incorporated into a chemical entity that is adapted for uptake into the target cells, in which case the short-range Auger electrons exert their destructive effects directly on the DNA within the cell in which they are contained, and with minimal collateral damage to surrounding cells

An embodiment of the present invention is also directed to a method of treating a patient for a malcondition wherein intra-tissue delivery of a bioactive agent is medically indicated, using the inventive catheter array system, by emplacing the catheter guide template within or adjacent to the target tissue of the patient such that the guide template is immediately adjacent to tissues targeted for the intra-tissue delivery of the bioactive agent; then, inserting each of a plurality of catheters through the guide template such that each catheter is directed by a respective channel to a position within the target tissue to form the spatially defined catheter array; and connecting the liquid supply system to the base portion of each catheter such that pressurized liquid can be delivered through the catheter to the target tissue; and then supplying a liquid comprising a solution of the bioactive agent from the liquid supply system through a plurality of catheters into the target tissue by way of the ports.

The catheter array system can be deployed within the patient's tissues, for example, within a void left by removal of a brain tumor, such that the plurality of catheters intrude into the tissue surrounding the tumor excision site. Alternatively, the catheter array system can be deployed within tumor plaques, such as occur in certain ovarian cancers.

The entire system can be emplaced entirely within the patient's body, such that the liquid supply system and manifold, as well as the catheter guide template and the plurality of catheters, are disposed under the patient's skin. Alternatively, the liquid supply system at least can be disposed external to the patient's body.

To the extent that the catheter guide template comes in contact with body tissue, it is preferred that at least the surface of the guide template be biocompatible, as can be accomplished through the use of appropriate materials of construction. Likewise, to the extent that the liquid supply system is adapted to be disposed within the patient's body, it's exterior surfaces can be biocompatible.

An embodiment of the inventive method can include the administration of the solution of the bioactive agent at a variety of pressures, flow rates, and durations of administration. For example, the solution can be administered continuously, intermittently, at various rates, and for various periods of time.

A preferred bioactive agent is a radiological agent, which can be an Auger electron emitting isotope, for example ¹²³I or ¹²⁵I, which causes mostly short-range damage to tissues in which it is disposed, thus limiting undesired radiation damage to healthy tissues. The Auger electron emitting isotope can be part of a molecule adapted to be incorporated into the cellular structure of cancerous cells in the target tissue; for example, a nucleotide analogue can be radiolabeled to provide a bioactive structure suitable for use in the inventive method. ¹²⁵I-iodouridinedeoxyriboside (IUDR) is an example.

### BRIEF DESCRIPTIONS OF THE DRAWINGS

FIG. 1A is a longitudinal cross sectional view of a fixed catheter array.

FIG. 1B is a 3-dimensional view of the fixed catheter array of FIG. 1A.

FIG. 1C is another 3-dimensional view of the fixed catheter array of FIGS. 1A and 1B, but including a guide template, with catheter guide channels.

FIG. 2A is a longitudinal center cross sectional view of a catheter guide template with multiple catheter guide channels into which catheters have been preloaded.

FIG. 2B is a longitudinal cross sectional view of a catheter guide device comprised of multiple catheter guide channels. The catheters have been advanced from the catheter guide channels at the distal end of the catheter guide template.

FIG. 2C is an facial view of the distal end of the catheter guide template of FIG. 2B. The catheters have been advanced from the catheter guide channels.

FIG. 2D is a longitudinal cross sectional view of a catheter guide template of FIG. 2A.

FIG. 3A is a center longitudinal cross sectional view of a catheter guide template.

FIG. 3B is a center longitudinal cross sectional view of the catheter guide template of FIG. 4A, but with the catheter tips extended.

FIG. 4A is a center longitudinal cross sectional view of the catheter guide template of FIG. 4B, but with catheters emerging from catheter guide channels only on one side of the device.

FIG. 4B is a cross sectional view of a brain (B), with a tumor cavity (TC) after surgical removal of the tumor, and site of tumor recurrence (TR). The catheter guide template of FIG. 4A has been emplaced to cover the site of tumor recurrence.

FIG. 4C is a cross sectional view of a brain (B), with a tumor cavity (TC) after surgical removal of the tumor, and site of tumor recurrence (TR). A catheter guide template has been implanted to cover the area of tumor recurrence.

FIG. 5A is an expanded view of an embodiment a catheter guide template including a series of cross sectional disks.

FIG. 5B is an expanded view of the catheter guide device of FIG. 5A, but with the catheter tips in the extended position.

FIG. 5C is a center longitudinal cross sectional view of the distal end of the catheter guide template of FIG. 5A.

FIG. 5D is an afferent (“bottom”) view of the proximal end of the catheter guide template of FIG. 5A.

FIG. 6A is a longitudinal cross sectional view of an expandable catheter guide template with an inflation bag or balloon in a deflated configuration, with multiple catheter guide channels attached to a flexible membrane.

FIG. 6B is a longitudinal cross sectional view of an expandable catheter guide template with the inner balloon inflated.

FIG. 6C is a cross sectional view of an expandable catheter guide template with the balloon expanded and catheter tips extended from the catheter guide channels.

FIG. 7A is a longitudinal cross sectional view of an expandable catheter guide template in which the catheter channel guide tubes both guide the catheter placement and maintain a bow-like structure when deformed.

FIG. 7B is a longitudinal cross sectional view of an expandable catheter guide template in the expanded position with the distal ends of the catheter guide channels into close proximity of the treatment tissue.

FIG. 7C is a longitudinal cross sectional view of an expandable catheter guide template in the expanded position with the catheters in the extended position.

FIG. 8A is a surface view of a catheter guide template that can be formed by connecting a series of vertically oriented strips, each containing a row of catheter channel guides.

FIG. 8B is a surface view of a catheter guide template formed by connecting a series of horizontally oriented rings, each comprised of a strip, each containing a row of catheter guide tubes.

FIG. 8C is a surface view of a catheter guide template formed of catheter guide template strips assembled into a helical structure.

FIG. 8D is a back view of a module of the catheter guide template of FIGS. 8A, 8B, and 8C.

FIG. 9A depicts a catheter.

FIG. 9B depicts a catheter with two additional catheter apertures or ports on the side of the catheter.

FIG. 9C depicts a blunt ended catheter with two catheter apertures or ports on the side of the catheter.

FIG. 9D depicts a blunt ended standard catheter with three catheter apertures or ports on the side of the catheter.

FIG. 9E depicts a square catheter with a standard single aperture or port at the end.

FIG. 9F depicts a square catheter with a blunt tip and two apertures or ports on the side of the catheter.

FIG. 9G depicts a blunt curvilinear catheter with four side apertures or ports.

FIG. 9H depicts a standard catheter with a rounded tip and single aperture or port at the end.

FIG. 9I is a longitudinal cross sectional view of a catheter with a catheter tip bumper and a single side aperture or port.

FIG. 10A depicts a distal catheter with a blunt tip, three side apertures or ports and an expansion joint to prevent back diffusion.

FIG. 10B depicts a distal catheter with a blunt tip, three side apertures or ports and an short expansion (or bump out) to prevent back diffusion.

FIG. 10C depicts a conically shaped distal catheter with a blunt tip and three side apertures or ports.

FIG. 10D depicts a distal catheter with a blunt tip, three side apertures or ports, an expansion section to prevent back diffusion, and a flexible section to facilitate bending without undue stress to the catheter tip.

FIG. 11A depicts a catheter with a guide wire inserted to increase mechanical strength during implantation of the catheter into the target tissue.

FIG. 11B depicts a catheter with a catheter tip bumper and a guide wire inserted to increase mechanical strength during implantation of the catheter into the target tissue.

FIG. 11C depicts a distal portion of a catheter with a guide wire inserted to increase mechanical strength during implantation of the catheter into the target tissue.

FIG. 12A is a cross sectional view of round catheter tubing.

FIG. 12B is a cross sectional view of oval catheter tubing.

FIG. 12C is a cross sectional view of rounded square catheter tubing.

FIG. 12D is a cross sectional view of square catheter tubing.

FIG. 12E is a cross sectional view of rectangular catheter tubing.

FIG. 12F is a cross sectional view of hexagonal catheter tubing.

FIG. 13A depicts a bulb-shaped grooved catheter with a blunt tip, viewed from a longitudinal perspective. The apertures or portals, 4 shown in this view, are located within the grooves.

FIG. 13B depicts a bulb-shaped grooved catheter viewed in cross section through a segment between the ports or apertures.

FIG. 13C depicts a bulb-shaped grooved catheter viewed in cross section through a segment including the ports or apertures.

FIG. 14 is a side view of a catheter array system according to the invention, with an expanded view of a flow control device.

FIG. 15 is a perspective view of a catheter array system of the invention adapted to treat a tumor plaque.

### DETAILED DESCRIPTION OF THE INVENTION

An embodiment of the present invention is directed to a catheter array system for delivery of a liquid solution of a bioactive agent into a target tissue of a patient; the system comprising: a plurality of biocompatible catheters, each catheter comprising a linear or curvilinear hollow tube and being adapted for insertion into the body tissue, for remaining within the tissue for a period of time, and for delivery of the solution of the bioactive agent through the tube into the tissue; a catheter guide template adapted for guiding emplacement of each of the plurality of catheters into a tissue adjacent to the guide template to form a spatially defined catheter array within the tissue; and, a pressurized liquid supply system adapted for delivery of a liquid via a manifold to each of the catheters; wherein each catheter comprises a distal portion for insertion into the tissue, at least one port whereby the solution can pass from inside the hollow tube into the tissue, a median portion adapted to be directed for insertion into the tissue by the guide template, and a base portion adapted for connection to the manifold of the pressurized liquid supply system; the catheter guide template comprises a plurality of catheter guideway channels, each guideway channel being adapted to guide movement of one or more catheters through the channel for insertion into the tissue such that upon insertion of the plurality of catheters, the catheters can form the spatially defined catheter array within the tissue; and the liquid supply system comprises a pressurizer adapted to apply a pressure to the liquid solution, and, a manifold to deliver the liquid under pressure to the base portion of each of the plurality of catheters such that the liquid can pass through the hollow tube of each catheter into the tissue.

An embodiment of the present invention concerns surgically implanted drug delivery devices comprised of a plurality of catheters and a catheter guide template adapted to guide the implantation of such catheters into solid tissue, for example, brain tissue. The plurality of catheters, which are directed to form a spatially defined array within the tissue by means of the catheter guide template, are used to deliver bioactive therapeutic agents directly into tumors or tissues such as those that have been infiltrated by locally invasive, proliferating tumor cells. The bioactive agents include, but are not limited to radioactive compounds, cytotoxic and other small molecule drugs, antibodies, proteins, peptides, oligonucleotide aptamers, antisense oligonucleotides and siRNA. The catheter array system of the invention may be used to treat different types of locally advanced solid tumors. The treatment field may include the tumor itself and/or the tissues adjacent to the tumor. In certain situations, such as in patients with brain tumors, the treatment field may be located in the tissue adjacent to a post-surgical tumor resection cavity. Such tissue may be at risk for a tumor recurrence involving progressive invasion by proliferating tumor cells and tumor-associated neovasculature. In this situation, the treatment field includes the brain tissue adjacent to the tumor, and the treatment may be administered before and/or after tumor recurrence.

Local delivery of pharmaceuticals and radiochemicals is seldom performed. One reason is because the use of one or a few catheters results in either a very limited delivery zone based primarily upon diffusion or low flow rates, or a more extensive delivery zone based upon convection (bulk flow, higher flow rates), but with less accurate targeting in and around the tumor. The range and shape of the pharmaceutical delivery zone produced by a single catheter may have unacceptable variability due to tissue inhomogeneity within an organ, variable interstitial pressure, variable capillary density, uneven scarring, and/or variation related to the disease state (e.g. tumor fibrosis). In addition, the target area itself may be very large and irregularly shaped.

One method of overcoming the inherent problems of localized drug delivery is to use multiple catheters, each catheter being responsible for delivery to a small zone. Multiple catheters can then deliver overlapping zones of,the pharmaceutical to provide uniform and effective targeting in tissues of different shapes, sizes and densities. Of course this can be done by individually placing multiple catheters into the area of treatment. However, the individual placement of catheters is a tedious process with inherent difficulties in the exact relative placement of catheters. For example, see Bouvier G et. al., “Direct delivery of medication into a brain tumor through multiple chronically implanted catheters,” Neurosurgery, 20:286-291(1987).

The inventive catheter array system guides the placement of multiple catheters into a field of treatment where the individual sources of drug from each catheter determine an overlapping field of treatment. These devices utilize a network or array of catheters to expose the entire treatment field to an antineoplastic agent, radiopharmaceutical agent, or other pharmaceutical agent. A more uniform treatment field is possible since each individual catheter delivers the therapeutic agent to one part of the treatment field, also referred to as the sub-treatment field. Overlapping sub-treatment fields provide a complete and more uniform treatment field.

The inventive devices can be used to achieve orderly or evenly spaced catheter placement in a treatment field, within a much shorter time frame than can be achieved with individually emplaced catheters, and with a much higher degree of spatial accuracy, as is advantageous during surgery when the patient's body tissues, such as the brain, are exposed. Optimal positioning of catheters is important not only to ensure that the infusate gains access to the entire intended treatment field, but also to minimize exposure to uninvolved regions of the tissue or organ.

The plurality of catheters is adapted to remain within the tissue for a period of time. By this is meant that a catheter does not function merely analagously to a syringe needle, which is inserted into tissue, a material injected, and the needle immediately withdrawn. Rather, each of the catheters forming the array within the target tissue is left in place for a period of hours, or of days, or even of weeks, during which a solution of a bioactive agent, such as a radiological agent, is infused into the tissue at a relatively low rate. The catheters are adapted to deliver the solution of the bioactive agent under a certain amount of pressure, that is sufficient to enhance permeation of the tissue by the solution. Typically, resistance to liquid flow through tissue is relatively high, so absolute delivery rates are relatively low compared to a typical injection with a hypodermic syringe needle. Each of the catheters remains within the tissue for a period of time sufficient to infuse a target tissue volume with a desirable level of the particular bioactive agent being used in the particular situation.

The catheters are adapted to avoid backflow of infusate from the catheter track and into tissues at the point of catheter entry, and to avoid introduction of infusate into anatomical spaces beyond the treatment field, e.g. cerebral ventricles, leptomeninges or subdural space in the case of a brain tumor.

The spacing between the catheters forming the array; the relative orientation of the catheters with respect to each other within the array; and the orientation of the catheter array relative to the target tissue can be optimized to expose the entire target tissue to the drug containing liquid during the treatment period. The catheter array is adapted to minimize trauma to tissues in and around the treatment field during implantation of the device, during the treatment period, and during removal of the device.

Catheter arrays are created using guide templates to guide the implantation of catheter tips into the tissue in the spatially defined array. The guide templates determine the vector of each catheter and provide control over the depth of catheter penetration into the treatment field. A variety of guide templates are provided, each suitable for application to one or more target tissue types. In certain circumstances, the template may remain in place after implantation of the catheter array. In other instances, the template may be removed after implantation.

The system herein is adapted to provide orderly arrays of a plurality of catheters. The dimensions (length, internal and external diameters) of each catheter are determined functionally by several factors including the depth and diameter of the treatment sub-field; the density of catheters within the array; the intent to minimize damage to tissues; and optimal mechanical strength; and ease of implantation. The use of the inventive catheter array system provides an opportunity to implant drug delivery catheters at points inside of the brain tumor cavity, thereby focusing the treatment on regions of the brain that are most likely to harbor residual brain tumor cells (Hochberg, F. H., and Pruitt, A., Neurology, 30:907-911 (1980)) while avoiding trauma to regions beyond the tumor. Each catheter possesses features to minimize trauma to neural and vascular structures during and after insertion, for example, from within the tumor resection cavity. Use of modular catheter arrays provides an option to deliver therapeutic liquids into the treatment field using sustained infusions as well as a variety of pulsatile or otherwise episodic schedules of administration, including repetitive injections.

Catheter implantation into the target tissue and formation of the catheter array within the tissue is achieved by use of a catheter guide template, which can have a biocompatible surface. The guide template is adapted to guide the implantation of catheters in an orderly array with respect to each other and with respect to the tissue into which they are implanted. At least some of the catheters can be attached to a base prior to implantation, making a pre-formed array that may be directed by the guide template into the tissue. Alternatively, the catheters may be implanted under the direction of the guide template without being attached to a common base. Catheter guidance is accomplished by the use of catheter guide channels in the guide template. The channels provide a path to guide the position of the catheters during implantation; and are adapted to allow relative movement of the catheters through their respective channels during implantation. There can be features allowing the catheters to be locked in place after implantation, and in that case also to be unlocked when removal of catheters is desired.

The guide template may be left in place with the catheters following implantation, or the guide template may be removed after the catheters have been implanted. After implantation, the bioactive agent is discharged from the catheters into the surrounding tissue over a period of time, the bioactive agent being therapeutic for a malcondition of the patient. Preferably, the catheters are implanted within tissue in the vicinity of a tumor, such as an organ containing an advanced stage solid tumor. An example is the brain of a patient with a brain tumor. The catheter releases the bioactive agent such that the agent is concentrated in, and relatively evenly distributed throughout, the tissue that may contain cancerous cells, adjacent to the tumor or to the cavity remaining after surgical debulking of the tumor.

Certain types of cancer such as ovarian can present as tumor plaques on the peritoneum. Surgical resection is not always possible due to the numbers or locations or the plaques. Since these plaques are “thin”, an application of chemotherapeutic agent to a surface will penetrate the tumor tissue and destroy it. Thus, an embodiment of the invention is adapted to treat the surface of these tumors which in turn treats the whole of the tumor through diffusion of the pharmaceutical into the tumor. The catheter array devices are designed to place a large number of catheters in the area of the tumor. The size of the array can be quite large and even encompass the majority of the peritoneal cavity.

The implantation of catheter arrays is guided by the guide template with its guide channels, that can be positioned inside of the brain tumor cavity, by the direction of egress of the catheter from the catheter guide device and the structural rigidity of the catheter itself, which can be increased by the use of removable catheter guide wires. Accordingly, the invention provides methods for creating catheter arrays arranged in a variety of configurations and orientations relative to the surrounding brain tissue. In addition, the arrays have modular assembly features to allow delivery of therapeutic compounds to treatment fields with diverse 3-D shapes and sizes. Once the catheters array is implanted, therapeutic liquids may be introduced directly into the diseased tissues via a manifold that is connected to the plurality of catheters. Some of the devices described herein are adapted to permit changing the position of one or more catheters in the array during the course of the treatment.

In addition, the devices can be used in conjunction with image-based pretreatment planning. The inventive system can be used with accessories that provide for digitalized drug delivery to treatment fields having a wide variety of 3-dimensional shapes. In this context, digitized drug delivery means that the catheter arrays are arranged to supply a 3-dimensional treatment field that is congruous with a 3-dimensional treatment field that has been mapped using digital images obtained using computerized axial tomography (CT scans), magnetic resonance imaging (MRI), Positron Emission Tomography (PET scans), PET-CT, or other tissue imaging technologies. The 3-dimensional topography of the treatment field (target tissue) is defined prior to treatment, and may be revised during the treatment period to match the changing distribution of disease within the target tissue. Insertion of the catheters can be monitored by these same means. For example, radiopaque or paramagnetic substances can be included in at least some of the catheters, such as at the tips, to enable visualization of their positioning during the surgical procedure. In this manner, a pretreatment digital map of the target tissue can be used as an overlay to enable precise placement of the catheters during real-time monitoring of the surgery.

Alternatively, in pretreatment planning, a radiofrequency emitting probe can be used to determine stereotactic coordinates for emplacement of an object within the brain, which can be used in conjunction with, for example, a preoperative MRI scan to guide the exact emplacement of an object within a particular region of the brain. In an embodiment of the present invention, a radiofrequency emitting probe (RF probe) of this type can be used to guide the emplacement of individual catheters, catheter arrays, or the catheter guide template during the operation. For these purposes, the RF probe may be reversibly physically associated with a catheter, catheter array, or catheter guide template. The initial positioning and/or final emplacement of the catheters catheter arrays or catheter guide template may be guided using the stereotactic coordinates.

A catheter can be adapted to have affixed thereto, for example by a clip adapted for attachment and removal of a RF probe, the RF probe, which can be activated during the process of insertion of the catheter into the tissue. The point of RE emission is detected, and provides the stereotactic coordinates needed for precise emplacement of the catheter. Then, the RF probe can be detached from the catheter, and, optionally, used to emplace other catheters of an array. Alternatively, the RF probe can be used to guide the emplacement of a preassembled catheter matrix or array into the tissue. Alternatively, the RF probe can be used with a catheter guide template, enabling optimal positioning of the template prior to emplacing the catheters or catheter arrays into the tissue. The RF probe may be used to determine the optimal depth of insertion for each of the catheters or catheter arrays. Alternatively, the catheters or catheter arrays may be emplaced in the tissues after the position of the catheter guide template has been optimized under stereotactic guidance of the RF probe.

With fluid fluxes produced from each catheter, the use of the inventive catheter array system provides for more controlled and predictable drug delivery into solid tissues (e.g. brain), with minimal backflow, and with a reduced risk of delivering drugs into anatomic regions beyond the intended treatment field. The use of catheter arrays, each supplying a treatment sub-field, provides a method to more predictably and reliably distribute drugs into tissues with less risk of underexposing the “watershed zones” between adjacent treatment sub-fields. This reduces the guesswork that is invariably associated with the surgical placement of a small number of relatively large catheters into tissue surrounding the brain tumor resection cavity. Finally, the use of guide templates to create catheter arrays, is adapted for use in many types of solid tumors in addition to brain tumors, as well as in other therapeutic situations where it is medically indicated to suffuse a bioactive agent into a defined volume of tissue at a relatively uniform concentration throughout. For example, as mentioned above, malconditions involving tumor plaques, such as ovarian cancers wherein plaques for on the peritoneum, can be treated using inventive catheter arrays adapted to cover relatively large, relatively flat tissue surfaces, wherein the plurality of catheters can be adapted to penetrate the plaque to relatively shallow depths compared to, for instance, the depths to which catheters could be implanted in treating tissue surrounding an excised brain tumor.

The “target tissue” refers to the diseased tissue into which the catheters are implanted. The “treatment field” is the 3-dimensional domain of tissue to be treated with the entire catheter array. The treatment sub-field is the 3-dimensional domain of tissue supplied by a single catheter in the catheter array. The treatment field and target tissue can be the same.

The “solution of the bioactive agent” is any flowable composition containing a substance (a therapeutic substance) deemed to be useful in the treatment of a disease. The solution may contain one or more therapeutic substances, including but not limited to radioactive compounds, small molecule drugs, antibodies, proteins, peptides, oligonucleotides. The therapeutic substance may be dissolved (solution) or suspended (emulsions, miscelles, liposomes, particles, etc) in the therapeutic liquid. As the term is used herein, a “solution” of a bioactive substance also includes a suspension or a dispersion that is suitable for infusion by way of the catheters. Once the solution enters the tissue, it is referred to as the “infusate.”

“Catheters” are hollow or tubular structures, which are implanted directly into the treatment field. A solution of a bioactive agent is introduced into the target tissue (treatment field) via the catheters. Catheters are hollow, having a lumen or central channel through which the solution flows from the liquid supply system into the tissue. A catheter comprises a tip, and one or more openings, apertures or ports at or relatively near the tip, or on any portion of the catheter adapted to be in direct contact with the tissue. A catheter may be linear or curvilinear, and is adapted for implantation into solid tissue of a patient. The catheter may comprise one or multiple thick segments, rings or bulges on the outside of the shaft to reduce backflow around the catheter track and thus promote uptake of the infusate into the tissue. The catheter may further comprise a non-cutting rounded tip to minimize trauma to tissues during implantation.

The base of the catheter is connected via a manifold to the source of the pressurized liquid containing a pharmaceutical or radiochemical agent. The base of the catheter provides the route for delivery of liquid to the distal end of the catheter, which resides within the tissue after implantation.

Each catheter has a tip that pierces the target tissue. The catheter tip may have an aperture or port (open end) or it may be plugged (closed end). The tip and nearby sections of the catheter can also include ports adapted for emission of the solution. The therapeutic liquid flows through the lumen out of the aperture or port and/or port(s) into the treatment field. A catheter may contain one or more apertures or ports. Ports may be located a various places on the catheter, including the tip and/or the sides.

The catheter tips may be equipped with catheter tip bumpers intended to minimize tissue trauma as the catheter tip pierces the target tissue during insertion. Catheter tip bumpers may be comprised of a hard substance such as metal or a soft polymeric material. Bumpers can have a blunt contour to provide non-cutting dissection of the target tissues. These features reduce the risk of damage to blood vessels and nerve tracts in the path of the catheter tip. Catheters may include expanded sections, bulges, intended to minimize backflow of treatment fluid flowing from the apertures or ports.

The “catheter track” is a channel formed in the tissue as the catheter is advanced. The catheter track surrounds the catheter following implantation.

A catheter “base” is connected to the source of the solution by means of a manifold. The catheter tip enters directly into the treatment field, and maintains contact with the target tissue, whereas the catheter base does not enter the target tissue. The catheter base may come into contact with tissues outside of the treatment field.

“Flexible joints” may be included in the catheter tubing to reduce potential traction on the target tissues at the point of catheter entry. Flexible joints may be included anywhere in the catheter tubing system or catheter. “Expansion joints” allow compression or expansion of the catheter along its primary linear axis.

“Catheter arrays” are comprised of two or more catheters arranged in a specific configuration. Catheter arrays can be parallel or radial (positive or negative) arrangements of catheters, but may have alternative configurations as described below. The simplest catheter array has a brush-like configuration with at least two catheters.

The catheter guide template with its guide channels accurately guides each catheter into its defined position within the tissue during implantation. A variety of catheter guide templates are described in below. Catheter guide templates (a) provide pre-determined spacing between the catheters within a catheter array; (b) determine the relative orientation of the catheters with respect to each other as they enter the treatment field; and (c) determine the relative orientation (i.e. vector) of the catheters with respect to the target tissue. The guide template is comprised of two or more catheter guide channels or catheter guide tubes into which the catheters are inserted for implantation. Catheter “guide channels” provide defined paths for the catheters to follow during implantation, and are adapted to allow relative motion of the catheters through the respective channels during catheter implantation. During implantation, the catheter tips emerge from the distal or efferent end of the catheter guide device. The operator controls implantation of the catheters at the proximal or afferent end of the catheter guide template. The efferent and afferent aspects of the catheter guide template may be designed with differently in each type of template device. The catheter guide template provides at least two catheter guideways that determine the relative orientation of two catheters with respect to each other an with respect to the tissue into which the catheters are inserted. Preferably larger numbers of catheters are preferred, for example a guide template can provide for emplacement about 10, or about 20, or about 30 individual catheters.

Catheter guide channels are linear, curvilinear or dog-legged (i.e. bent) passages, tubes or holes that serve the purpose of directing individual catheters to a site of egress from the catheter guide device. In addition, these passages, channels give the catheter a vector upon egress from the catheter guide device.

The system may have as few as 2 and as many as several hundred individual catheters (preferably between 5 and about 50). The base ends of the catheters are attached to manifold that is connected to a portal tubing system into which the therapeutic liquid is introduced under pressure. The template channels may be arranged in a defined pattern located on the afferent aspect of the template, the “catheter hub.” The operator can control implantation of the catheters by manipulating the catheter tubes at the catheter hub. After implantation, the afferent ends of the catheters are connected to a catheter manifold.

Base or afferent portions of the catheters can converge upon a common chamber referred to as the manifold. The device can provide a mechanism to connect the afferent sections of the catheters to the manifold. The manifold can then be connected to the portal tube, into which the therapeutic liquid may be introduced. The portal tube may terminate outside of the body or beneath the surface of the body. The therapeutic liquid is introduced into the portal tubing system using a mechanical pump, osmotic pump, syringe, or any device capable of generating hydrostatic pressure. Preferably, the manifold is inside the body, but it may also be outside the body.

In to some embodiments the catheters, or the catheter tubes connected to the afferent ends of the catheters, or both, may be formed of a pliable or supple material. In this case firm but flexible catheter guide wires can be used to facilitate implantation. Catheter guide wires are inserted into the lumen of the catheter. Catheter guide wires may be removed or left in place after implantation.

The catheter guide template can be equipped with one or more inflatable balloons or other padding components to minimize displacement of the device after implantation. The catheter guide template balloon is adapted to maintain a snug fit, maintain catheter placement, and to reduce potential traction created by the movement of device components on the surrounding tissues. In some devices, a balloon may be used to compress the catheter arrays against the surrounding tissues. Balloons may be filled with air, fluid or gels.

There are various geometric variations for the relative vectors that the catheters take while penetrating the target tissue. One is an array of catheters that are all parallel, which allows for concurrent insertion of all the catheters.

Another is to have the direction of the distal catheters determined by catheter guide tubes or catheter guide channels, in which case the catheters can be inserted individually or in small sets. The guide channels allow an great variety of directions for individual catheters. However, the preferred orientation of the catheters, following emplacement with the guidance of the channels, is a parallel or radial pattern within the tissues.

The present invention will be described with reference to the attached drawings, which are given by way of non-limiting examples.

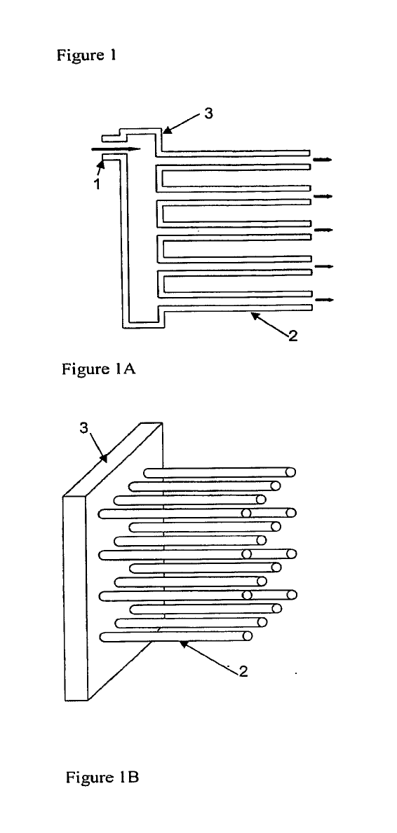

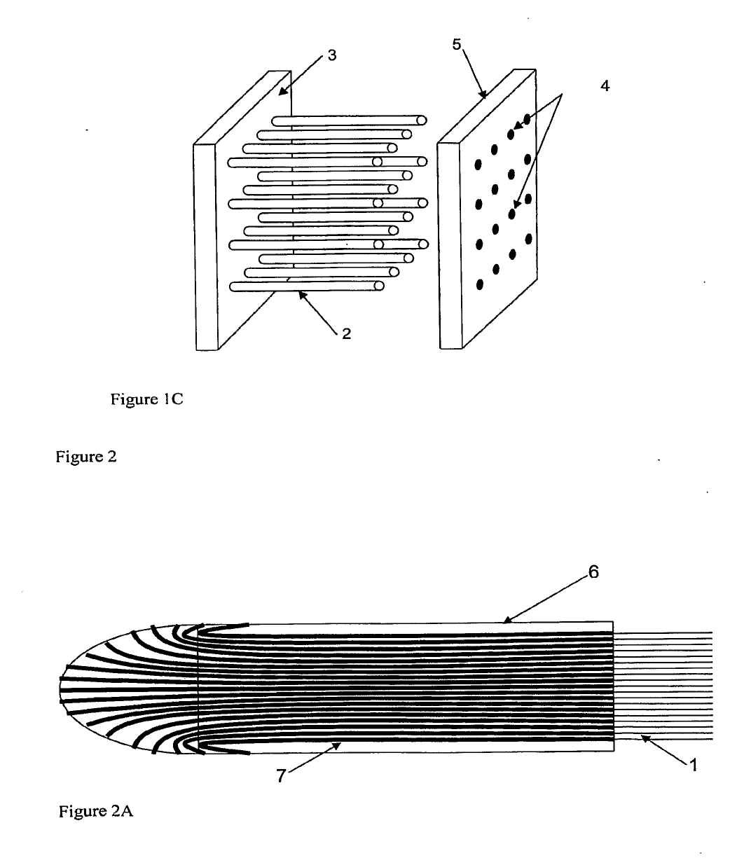

FIG. 1 illustrates a parallel array of catheters (2). The afferent (base) end of the catheter system (1) is joined via a connecting tube to a reservoir containing the pharmaceutical laden liquid (not shown). Under the force of hydrostatic pressure, the pharmaceutical solution is delivered to a base segment (3) that is connected to the catheters (2) from which the liquid is discharged into the tissues. The advantage to this design is ease of manufacture and ease of use as long as there is sufficiently large access to the site of implantation; however, the overall device may be rather bulky since the catheters are fixed in the extended position. If there are concerns that the device may dislodge from the site of implantation, it can be held in place by using a rigid afferent catheter (and affixing the afferent catheter on a solid support) or alternatively using a balloon inserted and preferably inflated with a viscous liquid (or a gas) to hold the catheter array in place. In an alternative design FIG. 1C also uses a catheter guide template, or template guide, to ensure a proper placement of catheters (by keeping the catheters parallel until after penetration of the tissue).

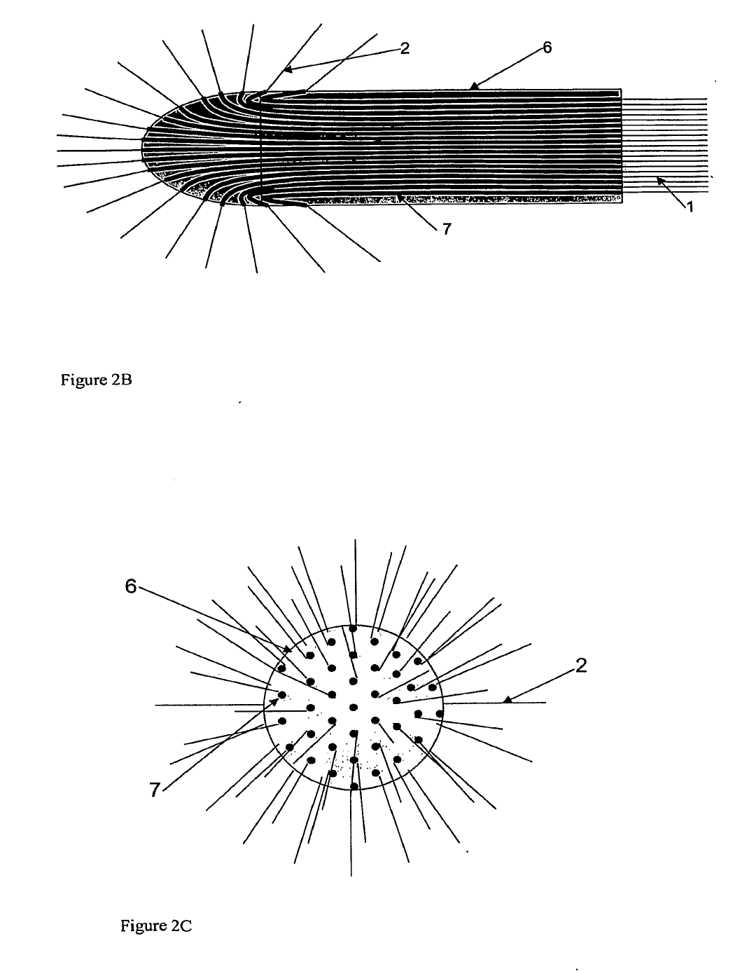

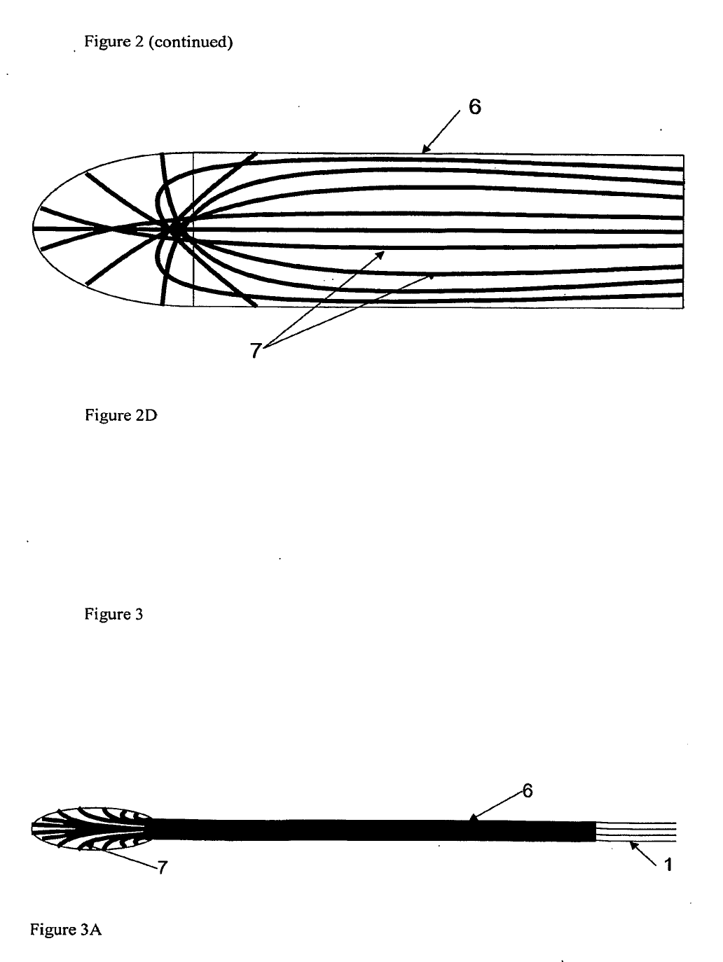

The preferred embodiment in FIG. 2 uses catheter channels within a rigid outer body. FIG. 2A is a longitudinal cross sectional view of a catheter guide template showing the template body (6), catheter channels (7) and afferent ends of the catheters (1). FIG. 2B is a longitudinal cross sectional view of a catheter guide template with the distal catheters extended (2). FIG. 2C is an illustration of the catheter guide template viewed from its efferent (distal) end with the catheters extended (2). The rigid outer body (6) serves as an attachment surface for the catheter guide channels (7) and also covers the individual catheter. The catheter guide channels (7) serve not only to bring the distal catheters (2) to a proper exit point on the surface of the catheter guide device, they also serve to give the exiting distal end of the catheter a vector of entry into the tissue to be treated. The catheters (2) are extended simply by pushing the afferent ends of the catheters (1) into the catheter guide template. Thus the advantages of this design are that (a) the distal catheters can be adjusted to various depths of penetration and (b) the distal catheters are not extended until the device is in its final position for treatment which reduces the possibility of tissue damage. Although the catheters can be flexible, FIG. 2D is an alternate design for the catheter guide channels wherein the arc in the tubing is minimized to prevent kinking in the catheter guide tubing and to allow for easier extension and retraction of the catheters.

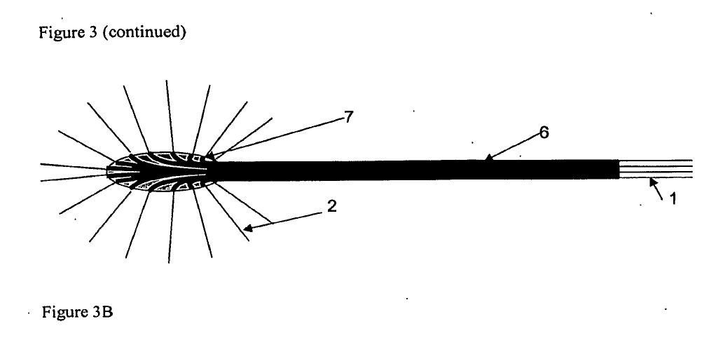

FIG. 3A is a longitudinal cross sectional view of a catheter guide template similar to FIG. 2A. In this case the afferent ends of the catheter guide channels (7) are bundled together to make a slimmer overall catheter guide template profile. This thinner design can aide in the placement of catheters into smaller cavities and also will help in the design of flexible catheter guide templates. This will allow the manufacture of “bendable” or flexible delivery systems to accommodate odd or irregularly shaped tumor cavities. FIG. 3B is a longitudinal cross sectional view of the catheter guide template with the distal ends of the catheters extended (2).

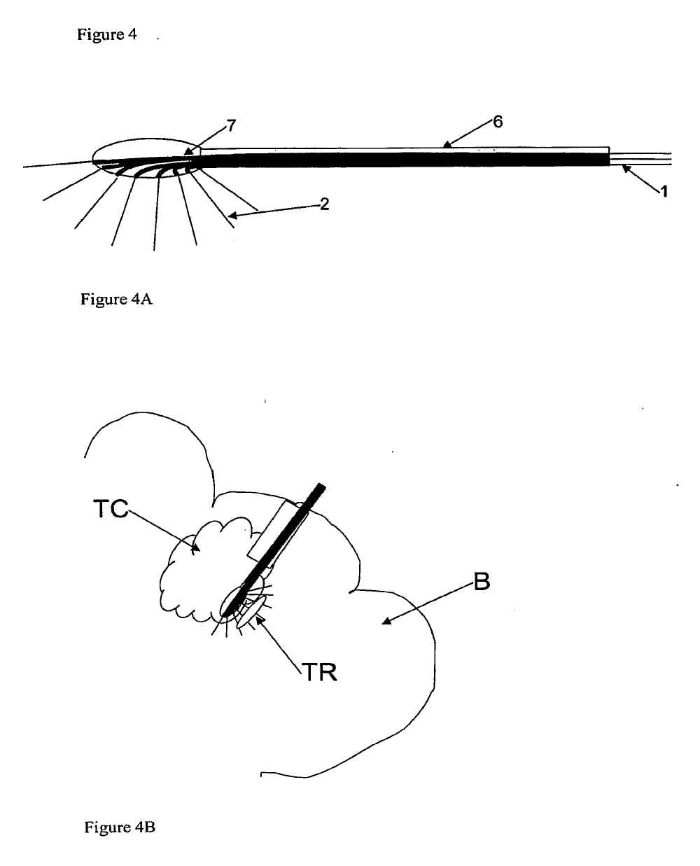

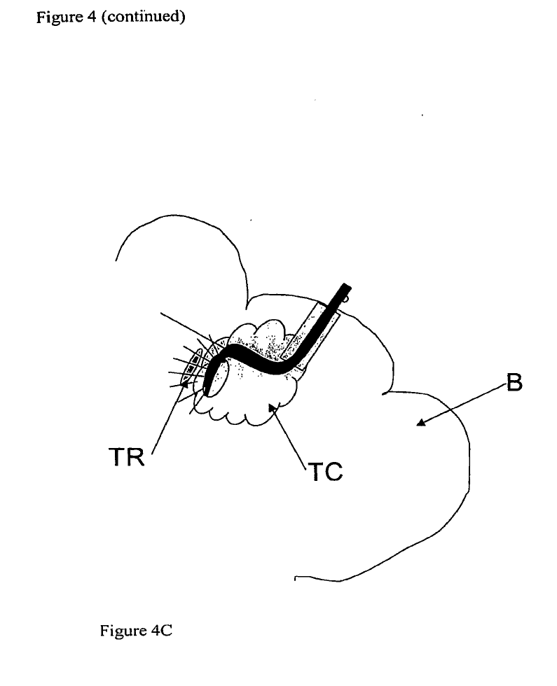

In addition to the flexibility of the design in FIG. 3A, FIG. 4A is an example of using only one half of the catheter guide channels in any design. This results in a design containing extendable and retractable catheters (2). In FIG. 4B is shown how this system could be emplaced to treat a tumor recurrence (TR) in a previously excised tumor cavity (TC) within the brain (B). FIG. 4C demonstrates the utility of having a bendable assembly of catheter guide channels within the template. In practice any number of catheters from one, to two, to 10%, to 90% (instead of the 50% presented in these figures) of the catheters could be extended in a variety of patterns to cover the required treatment field.

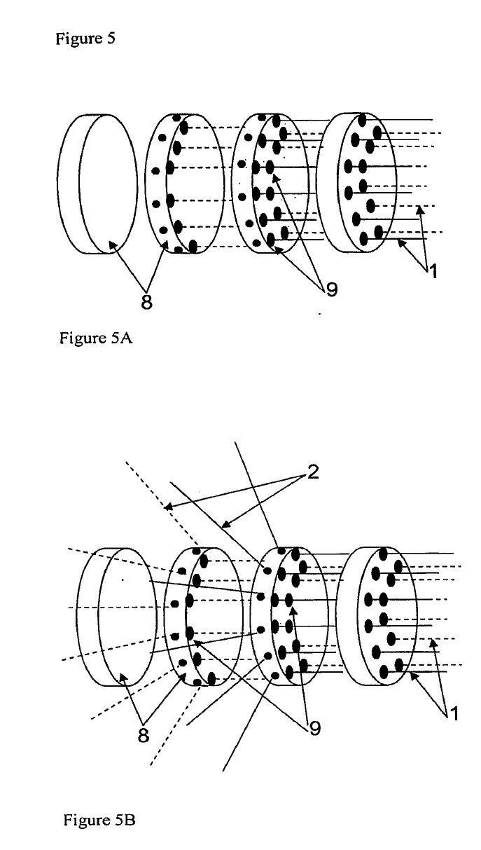

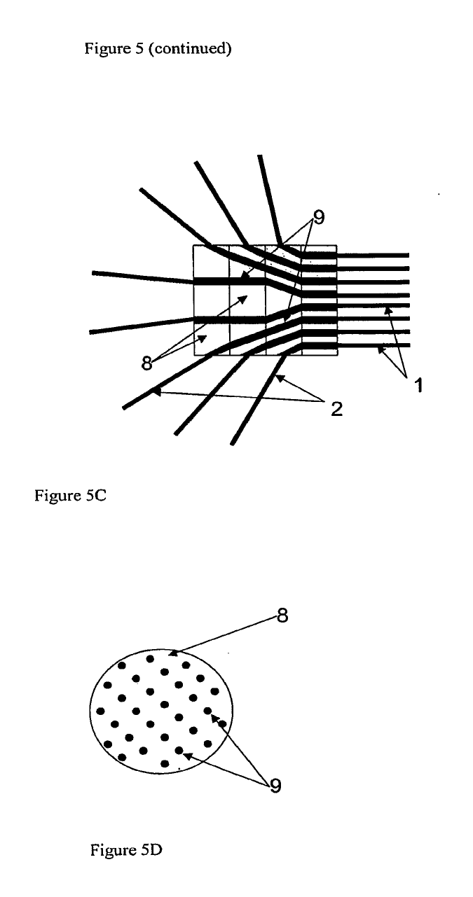

The spatial orientation of catheter guide channels is established by a fixed 3-D configuration (e.g. straight, curvilinear, bent) of each channel in the catheter guide device. The orientation of catheter guide channels may be established by modular assembly of channels thereby achieving a variety of configurations. For example, catheter guide channels (9) may be drilled or molded into the disks (8) that are assembled into a catheter guide device. The disks are assembled such that the holes are aligned to form the channels that determine the place of exit for the each distal end of a catheter and its directional vector relative to the device and tissue. FIG. 5A is an expanded view of a four disk (or plate) design and FIG. 5B is the same design with the distal ends of the catheters extended (2). FIG. 5C is a longitudinal cross sectional view of the same design. The directional vector of the catheters changes as they are advanced through the guide channels, beginning at the afferent end of the channel, and terminating at the efferent end of the channel. A variety of angles or arcs of curvature may be achieved in the catheter guide channels using either a series of linear segments arranged at angles (i.e. dog-legged, as shown in FIG. 5C) or using curvilinear design (not shown here). FIG. 5D is an illustration of the catheter guide device viewed from its efferent (distal) end without the catheters extended.

An important feature of the catheter guide channel designs is that they can be customized to implant catheters at any angle desired. This includes but is not limited to catheters that cross (giving better anchorage), perpendicular penetration of catheters to tissue (to minimize depth of catheter tip penetration), penetration of catheter into tissue at an angle (e.g. to reach tissue sites distant to the catheter guide assembly), catheters parallel to each other, etc. An equally important feature of the catheter array design is that catheters can be inserted into tissue at different depths. Catheters can be inserted to different depths to help with delivery zone overlap, or to help in effective treatment of an irregular tumor resection margin. Although it is preferred that a single catheter emanate from each catheter guide channel, multiple catheters can extend from a single channel. For example two separate catheters from the same guide channel can penetrate to different depths, or two separate catheters from the same channel can have different inherent curvatures causing them to penetrate the target tissue at different places even though they emanate from the same channel. It is understood that by using all of these features the skilled operator can create a catheter array with a great variety of configurations which can be customized with different depths of penetration, different penetration vectors, and different catheter designs.

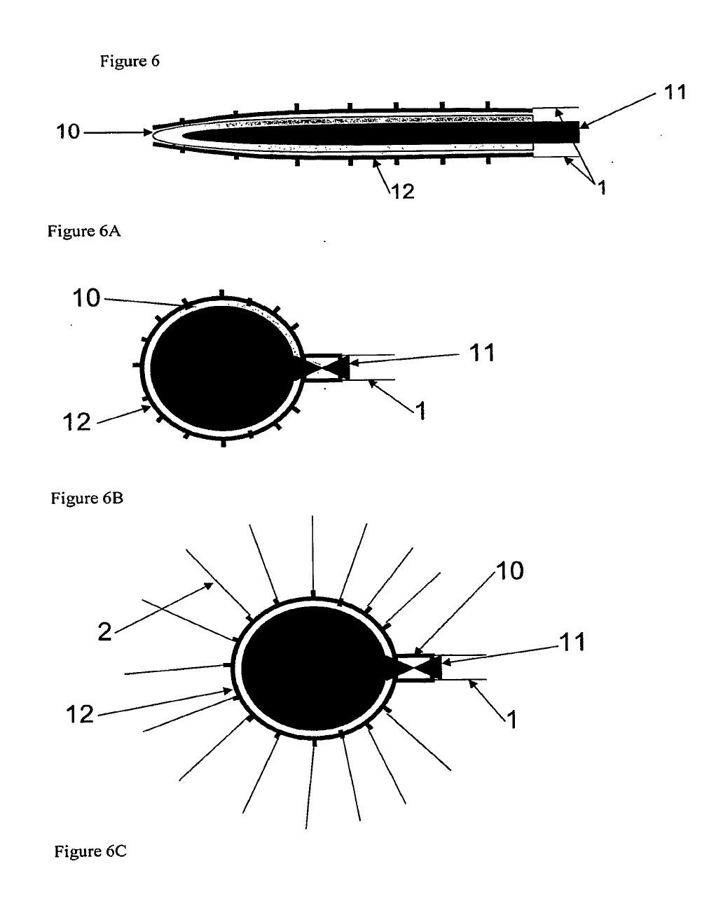

Expandable catheter guide templates can be adjusted to accommodate or “fill” the cavity left behind after surgical resection of the tumor. The surgery leaves a cavity that can vary in volume, shape, and depth of the cavity from the surface of the body. Having an expandable guide template allows the treatment of a wider variety of tumor cavities. FIG. 6 illustrates an expandable structure in which the catheter guide channels (12) are themselves flexible and attached to a membrane (10) (or attached to the inflation device itself). This design then allows the deflated version to easily enter the tumor cavity and then to be expanded (FIG. 6B) by filling with a fluid, gel or gas. Sealing the “balloon” (11) allows the catheter guide template to occupy the tumor cavity where the distal ends of the catheters (2) can be extended into the tissue (FIG. 6C). Note that depending on the flexibility of the catheters and the inflation device the catheter guide device need not be spherical and in fact could form a wide variety of shapes.

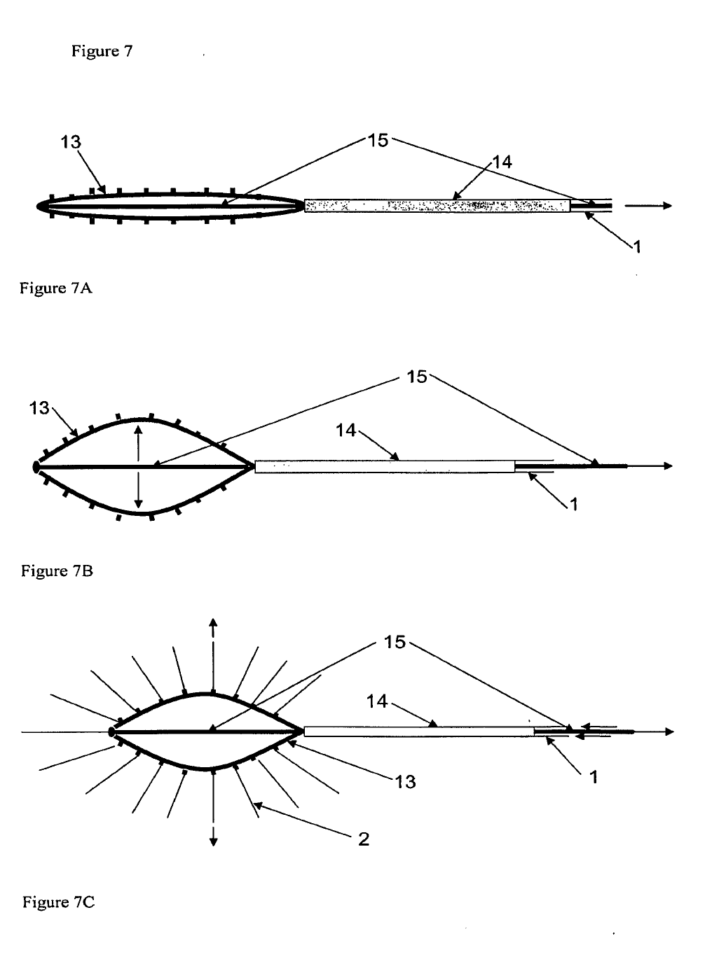

An alternate expandable catheter guide template uses catheter guide channels (13) that have a small amount of resistance to bending. Thus in FIG. 7A when the catheter guide channels are flexed by shortening a bow rod (15), the channels bow out as in FIG. 7B. The distal catheters (2) can then be extended after the template is in the tumor cavity (FIG. 7C).

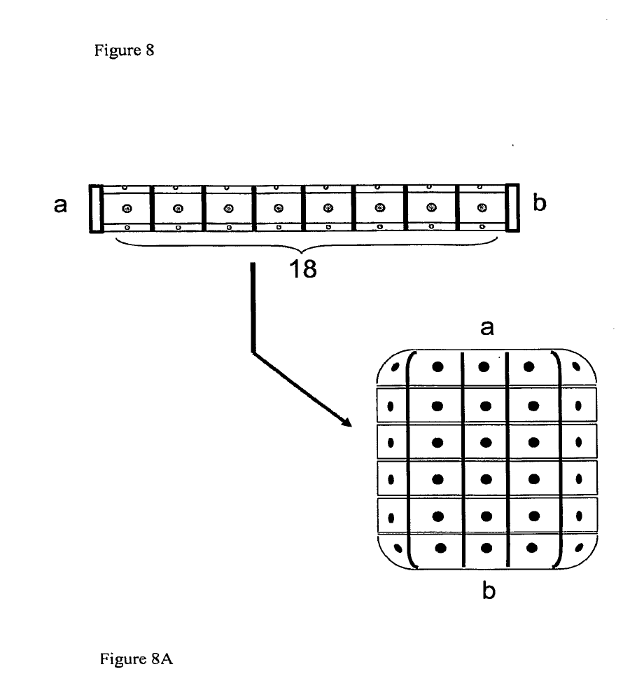

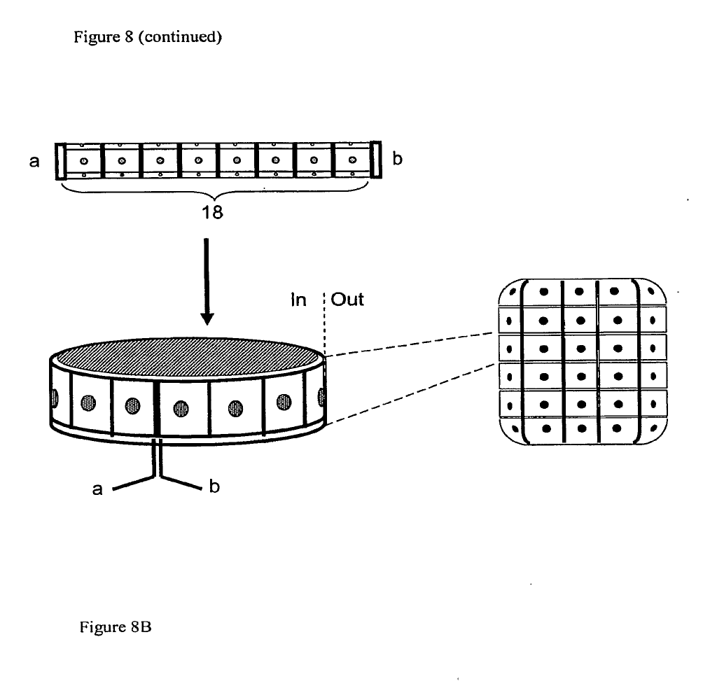

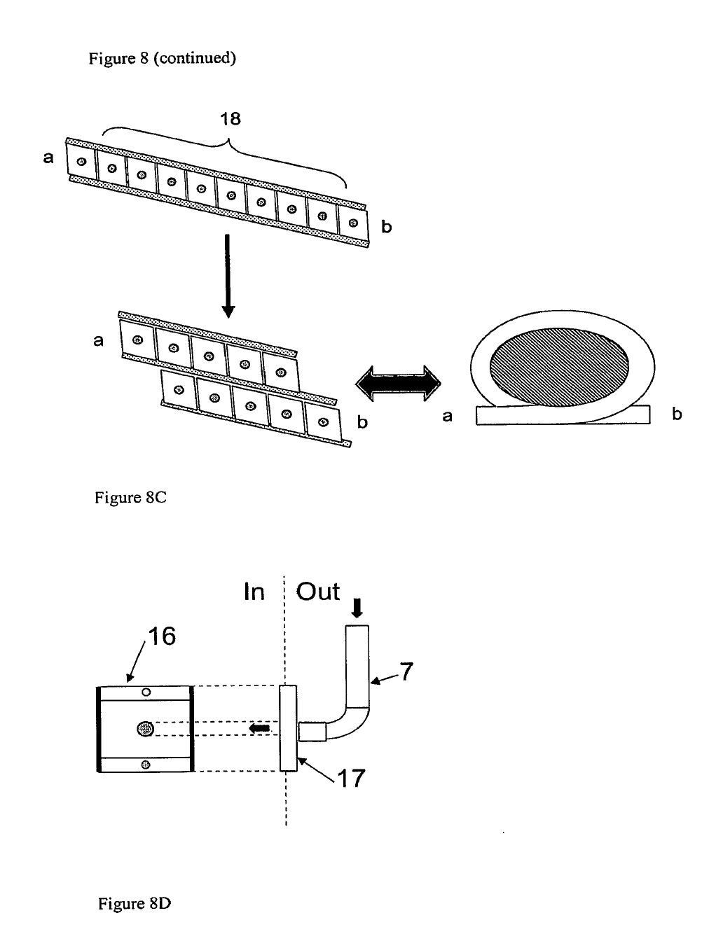

FIG. 8 illustrates a modular design that provides a catheter guide template with variable dimensions, i.e. may be assembled to fit different areas and circumferences. The basic unit is a strip of catheter guide holes that are linked together in a strip (18). The strip, or parts thereof, may be used itself as a simple template. Alternatively, the sides of these strips may be attached to each other by a linking mechanism, e.g. snaps, velcro, interlocking strips similar to zip-loc bags (see FIGS. 8A and 8B). Again this can be used to make catheter guide templates of different sizes or different circumferences, for example by linking them into a barrel shape. It is also possible to assemble a strip of catheter orifices into a helix (FIG. 8C) that assumes a cylindrical shape. The diameter of the resulting cylinder may be adjusted to the desired size (e.g. height and/or circumference) by sliding the helically arranged adjacent strips in either direction. A single unit of the modular catheter guide template is shown in FIG. 8D. The outside of the unit is illustrated (16). The catheter guide channels are attached to guide holes (17) inside of the barrel shaped catheter guide templates as illustrated in FIG. 8D. The afferent ends of the catheter channels can be bundled as they exit the device. Note that this design can be made of flexible but firm material to facilitate folding or deformation of the assembled device as necessary to provide an optimal fit into the tumor cavity prior to catheter extension.

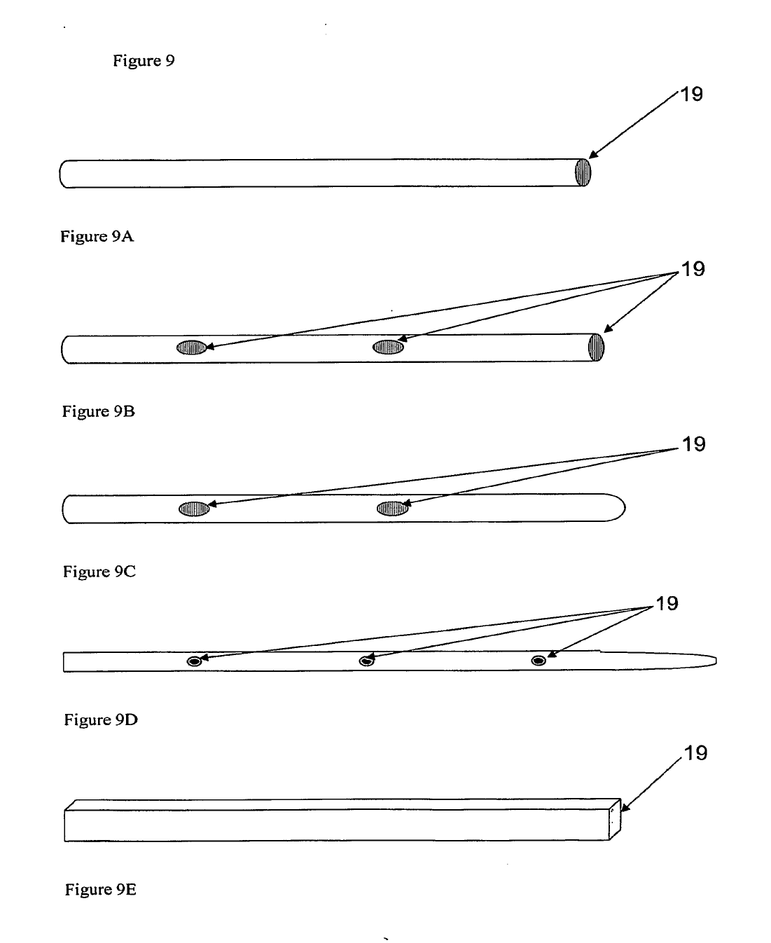

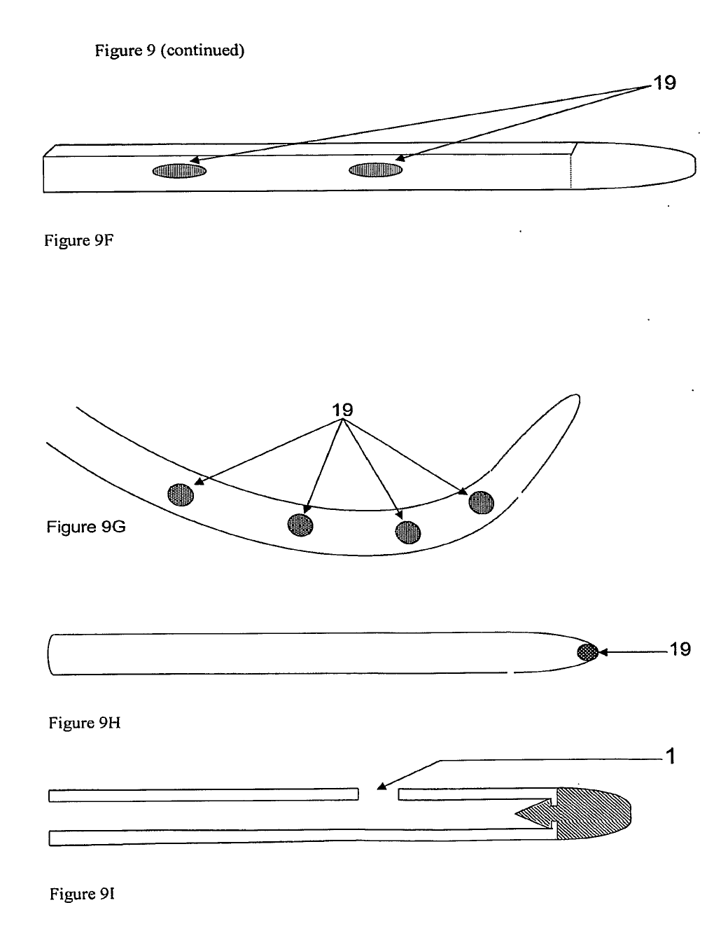

The catheters are designed with features to provide relatively uniform delivery of solutions of pharmaceutical agents. A single aperture or port as in FIG. 9A (19) is expected to deliver a roughly spherical pattern of drug assuming a uniform tissue density. Having multiple apertures or ports will increase the distribution of pharmaceutical agent into a pattern of distribution that is more ovoid than spherical (FIGS. 9B, 9C, 9D, 9F, 9G).

It is also important to minimize damage to tissues during distal catheter penetration into the treatment site. In a first iteration, a rounded catheter tip shown in FIG. 9H can be used to reduce trauma during insertion. In FIGS. 9C, 9D, 9F, and 9G blunt catheter tips without an apertures or ports are shown which can reduce the amount of damage during insertion. The material used to make the catheter may a different material than that used to form the tip, in order to minimize damage during insertion. Thus, FIG. 9I depicts a design using a catheter tip bumper made from a soft and/or pliable material that does not interact or stick to the tissue being penetrated. In addition, the use of flexible tubing may help to reduce damage by being deflected by blood vessels and other objects, although the tubing needs to be rigid enough to penetrate the target tissue. Catheters for use in delicate tissues such as the brain may be comprised of a soft material, whereas catheters for use in fibrous cancer tissues may be comprised of flexible, but mechanically strong, biocompatible polymers or metal.

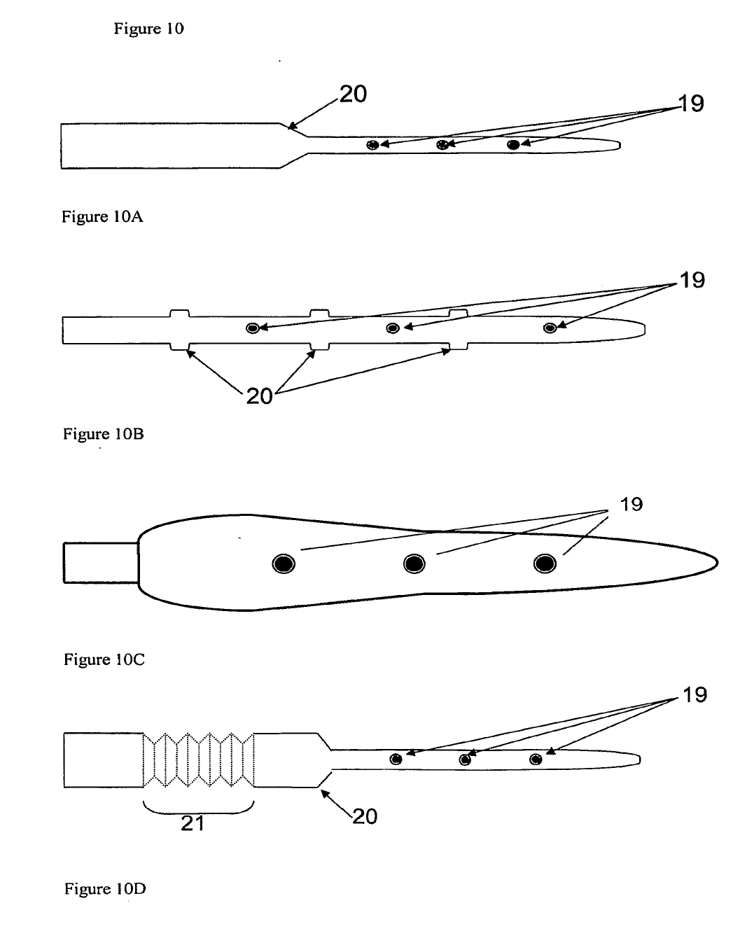

The catheter may have features designed to minimize or prevent back flow of the liquid pharmaceutical out of the insertion hole, the track, created by the catheter in the tissue. The use of a catheter extension section, and conically shaped catheters, are two methods of preventing back flow. In FIG. 10A the proximal end of the catheter is shown as having a larger diameter (20) than the distal end, thereby acting as a plug to prevent back flow. In FIG. 10B, multiple catheter expansions between the apertures or ports to facilitate uniform delivery from each drug delivery aperture or port. FIG. 10C illustrates a conical catheter design to prevent back flow. FIG. 10D shows a catheter with an expanded segment containing a “flexible joint” (21) to absorb torsional force applied to the afferent end of the catheter and thus minimizing any movement of the catheter tip inside of the tissue.

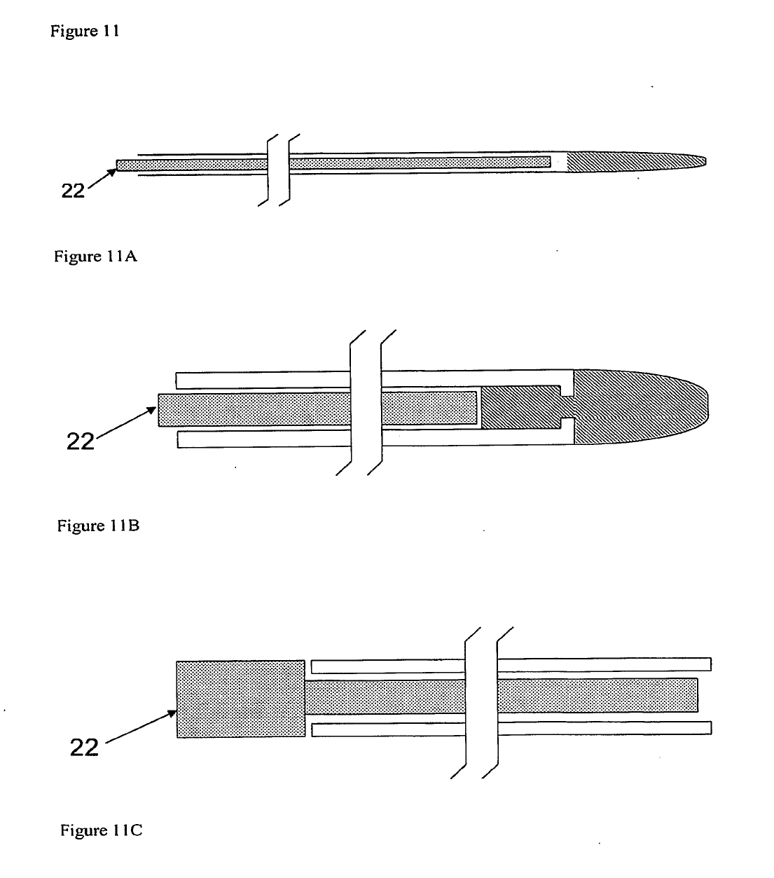

A catheter guide wire can be used to facilitate penetration of a catheter into the target tissue. A guide wire (22) are inserted into each respective catheter to increase mechanical strength during emplacement. FIG. 11A shows a guide wire placed in a blunt tip catheter; in FIG. 11B the guide wire is inserted into a catheter with a catheter tip bumper; and in FIG. 11C the guide wire is modified to be used in a catheter with the aperture or port at its terminal end. In each case the guide wire can be removed after insertion or left in place as long as there is adequate clearance around the guide wire to allow the pharmaceutical solution to reach the aperture or port.

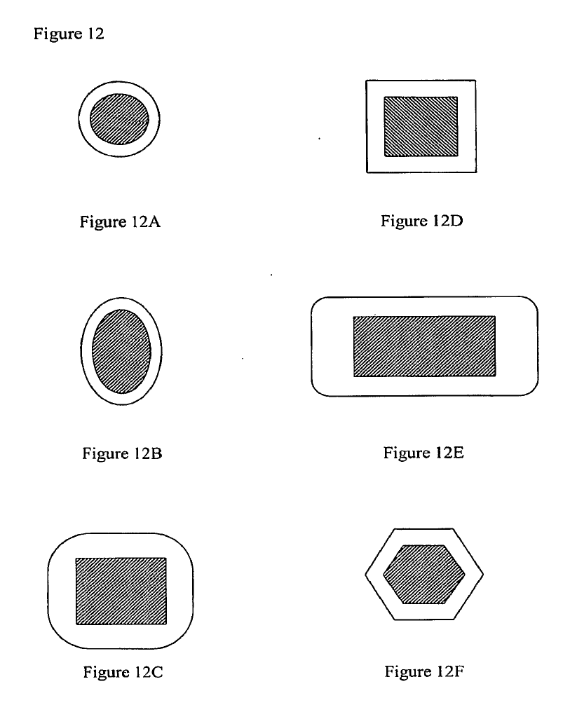

One additional method for increasing structural stability of the catheter tip during insertion into the tissue is to modify the shape of the tubing. FIG. 12A is an example of a round tubing design. FIG. 12B is an oval design that will have increased resistance to bending in the long axis of the oval while have a relatively easier time bending along the short axis of the oval. Similarly, a square design illustrated in FIGS. 12C and 12D will have an increased resistance to bending in planes that intersect with the corners of the tubing. This can be taken to higher levels as in FIG. 12F or even star shaped tubing to increase structural rigidity.

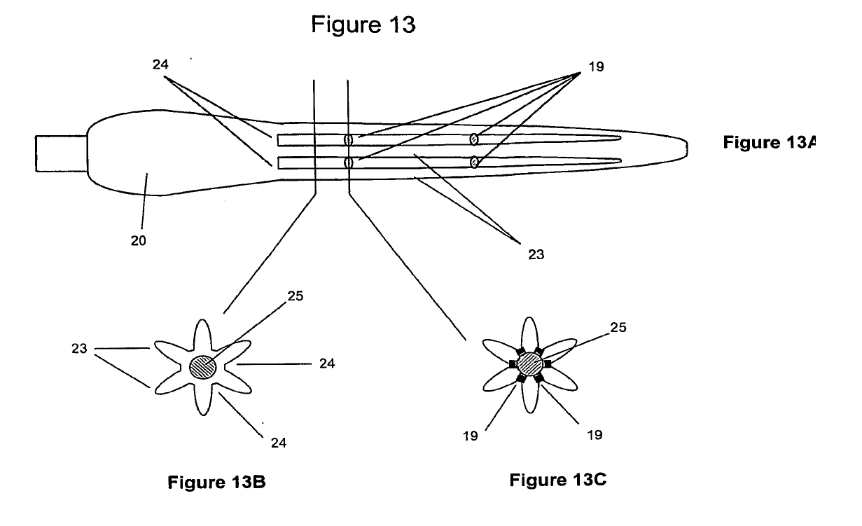

FIG. 13A illustrates a catheter with ridges (23) and grooves (24) oriented along the longitudinal axis of the catheter. This non-limiting example has six grooves and six ridges. The catheter ports (19) open into the grooves. There are two sets of six ports (only 4 are visible in FIG. 13A). This catheter design allows fluid exiting from the catheter portals to flow longitudinally in the grooves on the outside of the catheter, and thereby rapidly distributes the peak fluid pressure over the length of the catheter. A bulb-shaped blocking structure (20) is adapted to inhibit the back flow of the liquid expelled by the catheters out of the tissue through the track created in the tissue by emplacement of the catheter. FIG. 13B illustrates a cross section of the catheter illustrated in FIG. 13A; this cross section is through a segment between the ports, and shows the catheter lumen (25) and the star-shaped outer contour of the catheter. In FIG. 13, two grooves (24) and three ridges (25) are visible. FIG. 13C illustrates another cross section of the catheter illustrated in FIG. 13A; this cross section transects a segment of the catheter between the ports. The apertures or ports (19) are continuous with the lumen (25) of the catheter.

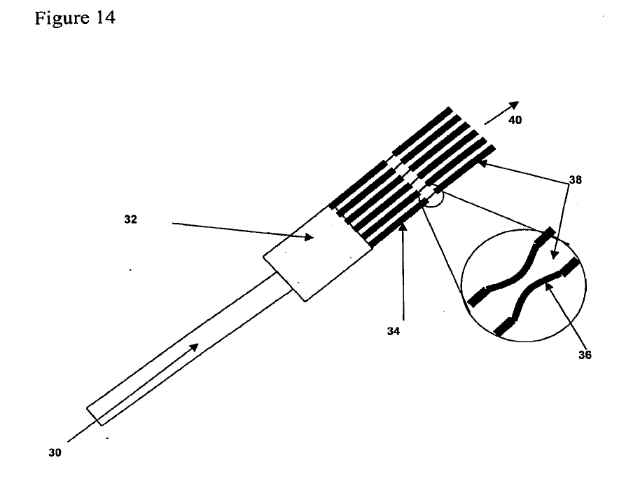

Referring to FIG. 14, an embodiment of a flow control device for each of a plurality of catheters is shown. Inflowing solution (30) from the liquid supply system (not shown) flows into the manifold (32) and from there into the bases (34) of each respective catheter. A flow control device (36), which can be a constricted section of the tube within each catheter, provides a regulating backpressure to equalize flows discharged from each of the distal ends (38) with their ports of the catheters. Thus the outflow (40) from the catheters is substantially equalized even in the presence of different backpressures on different individual catheters.

By the term “adapted to control a rate or volume of flow” is meant that by means of the flow control device, the individual flow from each catheter of an array can be altered from what it would be when implanted in a tissue without the presence of the flow control device. For example, fluid is supplied to all of the catheters of an array, but the backpressure experienced by each of the catheters can be very different, due to the inhomogeneity of the tissue in which the array can be implanted. Some catheters may encounter high backpressure, while others may experience virtually no backpressure. In such a situation, when there are no flow control devices present, the majority of the flow can be diverted into the catheter experiencing the lowest backpressure, thus diminishing the flow of the solution into the other catheters and from there into the tissue. In this way, the solution containing the bioactive agent can be wasted, or concentrated in a void where its presence has no therapeutic value. The flow control device, by providing backpressure through a constriction in the internal tube of each catheter, can limit the flow through catheters experiencing anomalously low backpressure, and thus lead to better dispersion of the solution of the bioactive agent throughout the target tissue. By the term “adapted to equalize a rate or volume of flow” is meant that the flow through each of the catheters is brought nearer to an equalized flow than would occur in the absence of the flow control device. Typically, it is desirable to control a rate or volume of flow through each of the catheters by attempting to equalize the rate or volume of flow, such that the solution is equally distributed throughout the target tissue and a small number of catheters that experience very low backpressures do not receive the bulk of the solution flow as a consequence.

Flow from one catheter into a void, or flow back along the catheter track into the resection cavity would in this way be expected to produce larger flow and a disproportionate delivery to this catheter and reduced flow to other catheters. In this embodiment of the invention, a flow control device is disposed between the manifold or pump and the catheter port or ports. The flow control device is a constriction in the diameter of the lumen inside of the catheter or at the junction between the catheter and manifold. Flow control may be regulated to varying degrees by using different degrees of constriction within the lumen of the catheters. Smaller constrictions provide larger pressure gradients, and therefore are expected to minimize the potential effects of unequal backpressure among the catheters. This flow control device will cause a build up of pressure in the manifold, and as long as the pressure is significantly higher than that in the catheter port, the result will be a constant flow through the catheter port regardless of local tissue backpressure. Each individual flow control device can also be adjusted to increase or decrease flow from each individual catheter. For example, an adjustable constriction can also allow individual catheters to be controlled in accordance to location and differences in backpressure. Alternatively, catheters with a fixed constriction of a particular size can be selected prior to implantation.

An embodiment of the invention concerns a method of treating a patient for a malcondition wherein intra-tissue delivery of a solution of a bioactive agent is medically indicated, using the inventive catheter array system, comprising: emplacing the catheter guide template within or adjacent to the target tissue of the patient such that the guide template is immediately adjacent to tissues targeted for the intra-tissue delivery of the solution of the bioactive agent; then inserting each of a plurality of catheters through the guide template such that each catheter is directed by a respective guideway to a position within the target tissue to form the spatially defined catheter array; and connecting the liquid supply system to the base portion of each catheter such that pressurized liquid can be delivered through the catheter to the target tissue; and then supplying a liquid comprising a solution of the bioactive agent from the liquid supply system through a plurality of catheters into the target tissue by way of the ports.

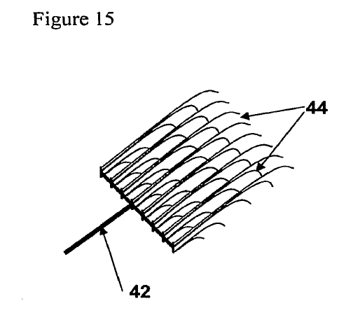

The method can include treatment of tissues surrounding a tumor excision site, for example in a brain tumor such as GBM, as discussed above in connection with certain embodiments of the inventive system. Use of the inventive system to create a defined spatial array of catheters within the tissue surrounding the tumor site which, as discussed above, is likely to contain residual cancerous cells and processes from an advanced stage localized tumor, can serve to deliver a therapeutic agent or a combination of agents to the tissue at a relatively uniform level throughout a volume of the tissue. Alternatively, the inventive method can comprise treatment of tumor for which no surgery or limited surgery indicated. For example, in certain ovarian cancers, tumor plaques can be formed on the surface of the peritoneum. Surgical resection is not always possible due to the numbers or locations or the plaques. An embodiment of the inventive method can use an inventive catheter array system are adapted to place a large number of catheters in the area of the tumor. Referring to FIG. 15, a catheter array system that can be used in treatment of a tumor plaque or plurality of tumor plaques is shown. Deep penetration not being needed to provide the solution to the thin surface plaques, a manifold (42) supplies the solution containing the bioactive agent or plurality of bioactive agents to a set of catheters (44) adapted to shallowly penetrate or treat the surface of the plaque and to cover a relatively large surface area (possibly including a large portion of the peritoneal cavity).

The fluid pharmacological agent may be discharged repetitively or intermittently from the catheters into the tissues as a result of temporary increases in the fluid pressure generated by the infusion pump. The increased fluid pressure may be instantaneous or brief in duration, thereby producing a rapid injection of the fluid pharmacological agent into the tissue. Alternatively, the pressure gradient may be more sustained, but not maintained continuously throughout the delivery of the agent, thereby producing one or more fluid waves that carry the fluid pharmacological agent into the tissue. In either case, the intervals between the repetitive or intermittent discharges of fluid may be brief (e.g. one second) or longer (e.g. several days). The latter are examples of pulsed delivery of the fluid pharmacological agent into tissue.

Alternatively, the fluid pharmacological agent may be discharged continuously from the catheters into the tissues as a result of a continuous pressure gradient generated and maintained by the infusion pump. In the latter case, the pressure gradient is maintained throughout the delivery of the agent, thereby producing continuous bulk flow of the fluid pharmacological agent into the tissue. The fluid pressure may be increased in one or more steps, increased continuously over at least part of the infusion period, or increased over all of the entire infusion period.

According to another embodiment of the invention, the fluid pharmacological agent may be discharged as a brief injection, a pulse, or as a more sustained infusion into the tissues, and then followed by an infusion of fluid that does not contain the fluid pharmacological agent. The fluid lacking a pharmacological agent may be introduced into the tissue by one or more instantaneous injections, one or more sustained waves of fluid movements, or by continuous bulk flow that is maintained by a constant pressure gradient.

The present invention also describes bioactive agents to be delivered using the catheter guide devices described above. The bioactive agent may be a radiochemical, chemotherapeutic agent or other small molecule, antibody, protein, peptide, oligonucleotide aptamer, antisense oligonucleotide or a small interfering RNA (siRNA).

An example of a radiochemical that may be delivered using the devices described herein is an Auger electron emitter, such as ¹²³I- or ¹²⁵I-iodouridinedeoxyriboside (¹²³IUDR or ¹²⁵IUDR). In this example, a radioactive ¹²³I- or ¹²⁵I-atom has been incorporated into a chemical entity, e.g. uridine deoxyribonucleoside, which is adapted for cellular uptake and incorporation into newly synthesized DNA in the target cells. In this example, target cells are defined as any cell in the treatment field engaged in DNA synthesis. Once incorporated into the chromosomes, the short-range Auger electrons are optimally located to exert their destructive effects directly on the DNA in the cell in which they are contained, and with minimal collateral damage to surrounding cells.

Numerous Auger electron emitting deoxyribonucleosides may be used, including but not limited to: 5-[¹²⁵I]-iodouridine 2′deoxyribonucleoside, 5-[¹²³I]-iodouridine 2′deoxyribonucleoside, 5-[¹²⁴I]-iodouridine 2′deoxyribonucleoside, 5-[⁷⁷Br]-bromouridine 2′deoxyribonucleoside, 5-[^{80m}Br-]-bromouridine 2′deoxyribonucleoside, 8-[¹²⁵I]-iodoadenine 2′deoxyribonucleoside and 5-[^{80m}Br]-bromoadenine 2′deoxyribonucleoside. In addition, alpha particle emitting deoxyribonucleosides may be used, including but not limited to: 5-[²¹³Bi]-bismuth uridine 2′deoxyribonucleoside and 5-[²¹¹At]-astatine uridine 2′deoxyribonucleoside.

In addition, it is understood that any prodrug of the above-mentioned nucleoside analogues can also be delivered using the devices disclosed herein. This includes a wide selection of phosphate and carbonyl esters involving the 5′ and 3′ hydroxyl groups on the ribose moiety of the nucleosides. For example, see US patent 20050069495 (Baranowska-Kortylewicz et al. Cancer specific radiolabeled conjugates regulated by the cell cycle for the treatment and diagnosis of cancer). Such prodrugs are hydrolyzed by nucleases, and in many cases by ubiquitous esterases, thereby releasing the active forms of such nucleosides, which after uptake by cells, are re-phosphorylated, recognized by cellular DNA polymerases and then incorporated into newly synthesized DNA. It is understood that a variety of chemical modifications of the nucleoside analogues containing the Auger or alpha particle emitting nuclides described above may be delivered using the devices disclosed herein. For example, nucleosides containing a 3′ deoxyribose may be incorporated at the terminal position of a growing strand of DNA prior to chain termination. Finally, it is understood that the ribose or base moieties of deoxynucleoside analogues such as ¹²³IUDR or ¹²⁵IUDR may be modified in numerous ways without necessarily interfering with their incorporation into newly synthesized DNA.

All publications, patents, and patent documents cited in the specification are incorporated by reference herein, as though individually incorporated by reference. In the case of any inconsistencies, the present disclosure, including any definitions therein, will prevail. The invention has been described with reference to various non-limiting examples and embodiments. However, it should be understood that many variations and modifications may be made while remaining within the spirit and scope of the present invention.

1-78. (canceled)

79. A catheter array system for delivery of a pressurized liquid solution of a bioactive agent into a target tissue of a patient; the system comprising a plurality of biocompatible catheters, each catheter comprising a linear or curvilinear hollow tube and being adapted for insertion into the body tissue and for delivery of the pressurized solution of the bioactive agent through the tube into the tissue, wherein the array system is adapted to be emplaced within the tissue to provide a spatially defined array such that the solution of the bioactive agent is delivered under pressure substantially uniformly to a volume of target tissue.

80. The system of claim 79 comprising a catheter guide template adapted for guiding emplacement of each of the plurality of catheters into a tissue adjacent to the template to form the spatially defined catheter array within the tissue.

81. The system of claim 79 comprising a preformed array of catheters adapted for emplacement of each of the plurality of catheters into a tissue individually or in groups wherein the orientation of the emplaced plurality of catheters into a tissue is determined by the relative orientation of catheters before and after implantation.

82. The system of any one of claims 79-81, wherein each catheter comprises a distal portion for penetration into the tissue, at least one port whereby the solution can pass from inside the hollow tube into the tissue, a median portion for transmission of the solution through the hollow tube of the catheter and a base portion adapted to receive the solution from a source thereof under a head of pressure.

83. The system of claim 80 wherein the catheter guide template comprises a plurality of catheter guideway channels, each guideway channel being adapted to guide movement of one or more catheters through the channel for insertion into the tissue such that upon insertion of the plurality of catheters, the catheters form the spatially defined catheter array within the tissue.

84. The system of any one of claims 79-81, further comprising a manifold to deliver the solution under pressure to each of the plurality of catheters such that the liquid can pass through the hollow tube of each catheter into the tissue.

85. The system of claim 84, further comprising a pressurized liquid supply system adapted for delivery of the solution via the manifold to each of the plurality of catheters, wherein the liquid supply system comprises a pressurizer adapted to apply a pressure to the solution.

86. The system of any one of claims 79-81 adapted to deliver a pressurized solution of the bioactive agent at a fluid flow rate that is sufficient to generate bulk flow or convection-enhanced delivery of the solution in the target tissues.

87. The system of claim 86 where the fluid flow rate into the target tissue is in the range of 0.5 ul/min to 15 ul/min.

88. The system of claim 81 wherein guidance of catheter insertion is provided by inherent physical characteristics or properties of the individual catheters to determine their final orientation within the tissues.

89. The system of any one of claims 79-81 wherein the plurality of catheters are adapted to remain within the tissue for a period of time.

90. The system of any one of claim 79-81 wherein the catheters and guide template, if present, comprise a biocompatible surface.

91. The system of any one of claims 79-81 further comprising catheter guidewires, each guidewire being adapted to fit within the hollow tube of a respective catheter, such that the guidewire provides rigidity and strength for insertion of the catheter into the tissue.

92. The system of claim 91 wherein each guidewire is adapted for subsequent removal from the emplaced catheter prior to delivery of the liquid through the catheter into the tissue.

93. The system of claim 91 wherein each guidewire is adapted to be left in place during delivery of the liquid through the catheter into the tissue.

94. The system of any one of claims 79-81 wherein the bioactive agent comprises a pharmaceutical or a radiological agent.

95. The system of any one of claims 79-81 wherein the bioactive agent comprises an Auger electron emitter.

96. The system of any one of claims 79-81 wherein the bioactive agent comprises a radiolabelled nucleoside or nucleoside analog comprising ¹²³I- or ¹²⁵I-IUDR or a ¹²³I-, ¹²⁵I-, ²¹¹At-, ²¹³Bi-, ^{80m}Br-, ¹²⁴I-, or ⁷⁷Br-labelled nucleoside analog, or any prodrug thereof.

97. The system of claim 95 wherein the Auger electron emitter comprises a radiolabelled nucleoside or nucleoside analog is an Auger electron emitting deoxyribonucleoside or analog thereof.

98. The system of claim 94 wherein the radiological agent is 5-[¹²⁵I]-iodouridine 2′deoxyribonucleoside, 5-[¹²³I]-iodouridine 2′deoxyribonucleoside, 5-[¹²⁴I]-iodouridine 2′deoxyribonucleoside, 5-[⁷⁷Br]-bromouridine 2′deoxyribonucleoside, 5-[^{80m}Br]-bromouridine 2′deoxyribonucleoside, 8-[¹²⁵I]-iodoadenine 2′deoxyribonucleoside, 5-[^{80m}Br]-bromoadenine 2′deoxyribonucleoside, 5-[²¹³Bi]-bismuth uridine 2′deoxyribonucleoside, or 5-[²¹¹A]-astatine uridine 2′deoxyribonucleoside.

99. The system of any one of claims 79-81 wherein the spatially defined catheter array comprises a parallel or a radial array of catheters disposed within the tissue.

100. The system of any one of claims 79-81 wherein the spatially defined catheter array comprises at least two sets of catheters wherein one catheter set penetrates the tissue in a parallel array and the second set penetrates the tissue in a second parallel array wherein the first and second parallel arrays are not mutually parallel.

101. The system of any one of claims 79-81 wherein the spatially defined catheter array comprises at least two sets of catheters wherein one catheter set penetrates the tissue to a greater distance than does a second catheter set.

102. The system of any one of claims 79-81 wherein the spatially defined catheter array comprises at least two catheters wherein each catheter penetrates the tissue to a distance that is unique for each catheter.

103. The system of claim 80 wherein the catheter guide template comprises a balloon and flexible guideways, wherein the balloon is adapted to be inflated after disposing the guide template on the tissue such that the inflated balloon shapes the guide template and positions the guideways for guiding insertion of the catheters through the guideways into the tissue to form the spatially defined array.

104. The system of claim 103 wherein the balloon is adapted to be inflated to substantially fill a tissue void within which the guide template is disposed such that the array of catheters can be emplaced within the tissue immediately surrounding the void.

105. The system of claim 80 wherein the catheter guide template comprises flexible guideways and further comprises a plurality of flexible ribs that are adapted to bend under pressure to conform to a tissue void within which the guide template is disposed such that the array of catheters can be emplaced through the guideways into the tissue immediately surrounding the void.

106. The system of claim 82 wherein at least some of the catheters have more than one port per catheter.

107. The system of claim 80 wherein at least some of the guideway channels are adapted to guide more than a single catheter.

108. The system of any one of claims 79-81 wherein at least some of the catheters have respective soft tips adapted to minimize tissue trauma upon insertion into the tissue.

109. The system of any one of claims 79-81 wherein at least some of the plurality of catheters further comprise blocking structures adapted to inhibit back flow of the liquid expelled by the catheters out of the tissue through an opening created in the tissue by emplacement of the catheter.

110. The system of any one of claims 79-81 wherein at least some of the plurality of catheters each further comprises a flexible joint.

111. The system of any one of claims 79-81 wherein at least some of the plurality of catheters have a cross-section that is non-circular.

112. The system of claim 111 wherein the non-circular cross-section is a square, triangular, pentagonal, hexagonal or star-shaped cross-section.

113. The system of any one of claims 79-81 wherein each catheter comprises a flow control device adapted to control a rate or volume of flow of the solution from a respective port of each catheter.

114. The system of claim 113 wherein each catheter comprises a flow control device adapted to equalize a rate or volume of flow of the solution from the respective port of each catheter.

115. The system of claim 85 wherein the liquid supply system is adapted to deliver constant or non-constant profiles over time of the flow rate or pressure of discharge of the liquid solution into the tissue.

116. The system of claim 115 wherein the profile of the flow rate or pressure of discharge of the liquid solution into the tissue is constant over time.

117. The system of claim 115 wherein the profile is an repetitive intermittent, episodic, pulsatile, curvilinear, or stepped flow rate or pressure of discharge.

118. The system of any one of claims 79-81 wherein the system and optionally a manifold and a pressurizer is adapted to be implanted substantially entirely within the body of the patient.

119. The system of any one of claims 79-81, adapted for administering a second bioactive agent for delivery to the target tissue.

120. The system of claim 119 wherein the radiological agent and the second bioactive agent are administered concurrently.

121. The system of claim 119 wherein the radiological agent and the second bioactive agent are administered non-concurrently.

122. A catheter adapted for use in the system of any one of claims 79-81.

123. A catheter guide template adapted for use in the system of claim 80.

124. A liquid supply system or manifold or combination thereof adapted for use in the system of claim 81.

125. A method of treating a patient for a malcondition wherein intra-tissue delivery of a solution of a bioactive agent is medically indicated, using the catheter array system of any one of claims 79-81, comprising emplacing the plurality of catheters within the target tissue forming the spatially defined catheter array such that the solution of the bioactive agent is delivered substantially uniformly to a volume of target tissue.

126. The method of claim 125 wherein the spatially defined array is created through operation of a guide template, or through operation of a stereotactic implantation system employing a radiofrequency probe disposed on a catheter to signal to the stereotactic implantation system a spatial position of the catheter within the tissue or relative to other implanted catheters or both.

127. The method of claim 126 wherein the guide template is emplaced within or adjacent to the target tissue and each of a plurality of catheters is inserted through the template such that each catheter is directed by a respective guideway to a position within the target tissue to form the spatially defined catheter array.

128. The method of claim 126 wherein the catheter guide template comprises a biocompatible surface.

129. The method of claim 125 wherein the array of catheters are emplaced into a tissue individually or in groups such that the emplacement of each of the plurality of catheters into a tissue is determined by the relative orientation of catheters before and after implantation.

130. The method of claim 125 further comprising connecting a liquid supply system to a base portion of each catheter of the spatially defined array such that pressurized liquid can be delivered through each catheter such that the solution of the bioactive agent is delivered substantially uniformly to a volume of target tissue, and then delivering the solution of the bioactive agent under a pressure from the liquid supply system through the plurality of catheters into the target tissue.

131. The method of claim 125 further comprising a sufficient fluid flow rate to deliver a pressurized solution of the bioactive agent at a fluid flow rate that is sufficient to generate bulk flow or convection-enhanced delivery of the solution in the target tissues.

132. The method of claim 131 where the fluid flow rate delivered under pressure is in the range of 0.5 ul/min to 15 ul/min.

133. The method of claim 125 wherein at least some of the plurality of catheters comprise catheter guidewires, each guidewire being adapted to fit within the hollow tube of the respective catheter, such that the guidewire provides rigidity and strength for insertion of the catheter into the tissue, wherein each guidewire is adapted for subsequent removal from the emplaced catheter prior to delivery of the liquid through the catheter into the tissue or is adapted to be left in place during delivery of the liquid through the catheter into the tissue.

134. The method of claim 125 wherein the spatially defined catheter array comprises a parallel array of catheters disposed within the tissue.

135. The method of claim 125 wherein the spatially defined catheter array comprises a radial array of catheters disposed within the tissue.

136. The method of claim 125 wherein the spatially defined catheter array comprises at least two subsets of the plurality of catheters wherein one catheter subset penetrates the tissue in a parallel array and the second subset penetrates the tissue in a second parallel array, wherein the first and second parallel arrays are not mutually parallel.

137. The method of claim 125 wherein the spatially defined catheter array comprises at least two sets of catheters wherein in the step of catheter insertion into the tissue, one catheter set penetrates the tissue a greater distance than does a second catheter set.

138. The method of claim 125 wherein the spatially defined catheter array comprises at least two catheters wherein each catheter penetrates the tissue to a distance that is unique for each catheter.

139. The method of claim 125 wherein the catheter guide template, if present, comprises a balloon and flexible guideways, wherein the balloon is adapted to be inflated after disposing the guide template on the tissue such that the inflated balloon shapes the guide template and positions the guideways for guiding insertion of the catheters through the guideways into the tissue to form the spatially defined array.

140. The method of claim 139 wherein the balloon is adapted to be inflated to substantially fill a tissue void within which the guide template is disposed such that the array of catheters can be emplaced within the tissue immediately surrounding the void.

141. The method of claim 125 wherein the catheter guide template, if present, comprises flexible guideways and further comprises a plurality of flexible ribs that are adapted to bow under pressure to substantially fill a tissue void within which the guide template is disposed such that the array of catheters can be emplaced through the guideways into the tissue immediately surrounding the void.

142. The method of claim 125 wherein at least some of the catheters have more than one port per catheter.

143. The method of claim 125 wherein individual catheter guideways contain more than one catheter per catheter guideway.

144. The method of claim 125 wherein at least some of the plurality of catheters further comprise blocking structures adapted to inhibit the flow of the liquid expelled by the catheters out of the tissue through an opening created in the tissue by emplacement of the catheter.

145. The method of claim 125 wherein at least some of the plurality of catheters further comprise a flexible expansion structure.

146. The method of claim 125 wherein at least some of the plurality of catheters have a cross-section that is non-circular.

147. The method of claim 146 wherein the non-circular cross-section is a square, triangular, pentagonal, hexagonal or star-shaped cross-section.

148. The method of claim 125 wherein a liquid supply system adapted to deliver constant or non-constant profiles over time of the flow rate or pressure of discharge of the liquid solution of the bioactive agent into the tissue is connected to each of the plurality of catheters.

149. The method of claim 148 wherein the profile of the flow rate or pressure of discharge of the liquid solution into the tissue is constant over time.

150. The method of claim 148 wherein the profile is an intermittent, pulsatile, curvilinear, or stepped flow rate or pressure of discharge.

151. The method of claim 125 wherein each catheter comprises a respective flow control device adapted to control or equalize a rate or volume of flow of the solution from the respective port or ports of each catheter.

152. The method of claim 125 wherein the bioactive agent an Auger electron emitter.

153. The method of claim 125 wherein the bioactive agent comprises a radiolabelled nucleoside or nucleoside analog comprising ¹²³I- or ¹²⁵I-IUDR or a ¹²³I-, ¹²⁵I-, ²¹¹At-, ²¹³Bi-, ^{80m}Br-, ¹²⁴I-, or ⁷⁷Br-labelled nucleoside analog, or any prodrug thereof.

154. The method of claim 152 wherein the Auger electron emitter comprises a radiolabelled nucleoside or nucleoside analog is an Auger electron emitting deoxyribonucleoside or analog thereof,

155. The method of claim 125 wherein the bioactive agent is 5[¹²⁵I]-iodouridine 2′deoxyribonucleoside, 5-[¹²³I]-iodouridine 2′deoxyribonucleoside, 5-[¹²⁴I]-iodouridine 2′deoxyribonucleoside, 5-[⁷⁷Br]-bromouridine 2′deoxyribonucleoside, 5-[^{80m}Br]-bromouridine.
